(19) United States
(12) Patent Application Publication
(10) Pub. No.: US 2016/0138115 A1
(43) Pub. Date: May 19, 2016

(54) METHODS AND CHARACTERISTICS FOR THE DIAGNOSIS OF ACUTE LYMPHOBLASTIC LEUKEMIA

(71) Applicant: MEMORIAL SLOAN-KETTERING CANCER CENTER, New York, NY (US)

(72) Inventors: Kenneth OFFIT, New York, NY (US); Sohela SHAH, New York, NY (US); Vajai JOSEPH, New York, NY (US); Kasmintan SCHRADER, New York, NY (US)

(73) Assignee: MEMORIAL SLOAN-KETTERING CANCER CENTER, New York, NY (US)

(21) Appl. No.: 14/900,112

(22) PCT Filed: Jun. 20, 2014
(86) PCT No.: PCT/US2014/043439

§ 371 Date: Dec. 18, 2015

Related U.S. Application Data
(60) Provisional application No. 61/837,516, filed on Jun. 20, 2013.

(51) Int. Cl. C12Q 1/68; C07K 14/47; C07K 16/18; G01N 33/50

(52) U.S. Cl. CPC C12Q 1/6886; G01N 33/5011; C12Q 1/6806; C07K 14/47; C07K 16/18; C12Q 2600/16; C07K 2317/33; C07K 2317/20

(57) ABSTRACT

Methods for identification of leukemia or a genetic predisposition to leukemia are provided that are particularly applicable to acute lymphoblastic leukemia (ALL). A novel heterozygous germline variant, c.547G>A (p.Gly183Ser) in the octapeptide domain of PAX5, is used to identify those individuals with an enhanced risk or predisposition to ALL.

This application is being filed on 20 Jun. 2014, as a PCT International Patent application and claims priority to U.S. Provisional patent application Ser. No. 61/837,516, filed Jun. 20, 2013.

### BACKGROUND OF THE INVENTION

1. Field of the Invention

Methods for identification of leukemia or a genetic predisposition to leukemia are provided that are particularly applicable to acute lymphoblastic leukemia (ALL). A novel heterozygous germline variant, c.547G>A (p.Gly183Ser) in the octapeptide domain of PAX5, is used to identify those individuals with an enhanced risk or predisposition to ALL.

2. Description of the Related Art

Acute lymphoblastic leukemia (ALL) constitutes a large proportion of pediatric hematologic malignancies. Of the estimated 6,050 cases of ALL in the USA, 1440 with ALL are expected to die due to cancer. Although recent care and management of these cancers have advanced to reduce mortality rates in recent years, other complications such as long term effects and the cost of care remains a significant challenge. There currently is no standard screen for leukemia. Just as these diseases are classified by their lineage differentiation, similar efforts have also made possible classification of tumors based on certain common chromosomal abnormalities. Knowledge about genetic mutations that predispose to cancers can greatly enhance surveillance, and risk-management. To date, no such variants are known for ALL.

No heritable genetic events have been described for ALL within the PAX5 gene. Other loci relating to ETV6-RUNX1 (PMID: 22076464) have been implicated in ALL susceptibility, but not alteration to the PAX5 locus as described herein.

There are many somatic PAX5 genomic alterations previously described in the leukemic cells of patients with ALL, which were explicitly absent from their native germline DNA. The present invention specifically describes a heritable variant within the constitutional DNA of individuals.

### SUMMARY OF THE INVENTION

The present invention relates to the methods for identification of leukemia or a genetic predisposition to leukemia. This invention is based on the identification of various genetic abnormalities particularly associated with ALL. These various abnormalities can be used to identify those individuals with an enhanced risk or predisposition to ALL. The invention provides discovery of compositions comprising single nucleotides that are altered in individuals within a gene PAX5, which may increase the risk of developing ALL. The specific invention describes a genomic PAX5 DNA mutation that changes a specific amino acid 183 from a glycine to a serine. Specifically, a novel heterozygous germline variant, c.547G>A (p.Gly183Ser), in the octapeptide domain of PAX5, is disclosed herein. The resultant protein is shown to have a weaker effect in regulating key B-cell specific molecular targets CD19 and CD79A which are some of the important differentiators of mature and immature B-cells. The genomic alteration is useful as a biomarker for enhanced surveillance and risk assessment.

It has been discovered that there is a significant association between the mutated residue and the cytogenetic finding of chromosome 9p abnormalities. In this subset of patients the residue was found to be mutated more frequently, indicating a population of patients who would benefit from germline mutation screening. The invention can also be used in a setting of prenatal screening and postnatal screening of germline mutation carriers of the susceptibility allele. This is very significant considering the role of in utero origin of some cases of pediatric ALL.

In some embodiments, methods are provided for characterizing by whole genome sequencing, methylome, transcriptome and proteome, a sample obtained from an individual human, detecting in the sample presence or absence of a mutation in the PAX5 gene or its encoded protein, wherein the presence of the mutation indicates that said individual has an increased likelihood of responding to a therapy that targets proteins encoded by genes usually repressed by the normal PAX5 or interferes with proteins upregulated by aberrant PAX5 in tumor cells.

In some embodiments, a method for identifying a human subject having an elevated risk of developing pre-B cell acute lymphoblastic leukemia (ALL) is provided, the method comprising (1) amplifying a nucleic acid comprising a region of the PAX5 coding sequence of SEQ ID NO: 1 in a tissue sample from said human subject, wherein said amplifying uses a primer set comprising a forward primer and a reverse primer, wherein said forward primer binds to a complement of said PAX 5 coding sequence at a position 5′ to codon 183 defined by nucleotides 547-549 of SEQ ID NO: 1 and wherein said reverse primer binds to said PAX 5 coding sequence 3′ to said codon 183; and wherein said amplifying generates an amplicon comprising PAX 5 codon 183, and (2) determining in said amplicon the amino acid encoded by said codon 183, wherein if said codon 183 encodes an amino other than glycine, said human subject has an elevated risk of developing pre-B cell ALL. In some embodiments, the codon 183 encodes an amino acid other than glycine, or alanine. In some embodiments, the codon 183 encodes an amino acid other than glycine, alanine or cysteine. In some embodiments, codon 183 encodes an amino acid other than glycine that is serine.

In some embodiments, a method for identifying a human subject having an elevated risk of developing pre-B cell acute lymphoblastic leukemia (ALL) is provided, the method comprising (1) amplifying nucleic acid comprising a region of a PAX5 gene sequence of SEQ ID NO: 4 in a tissue sample from said human subject, wherein said amplifying uses a primer set comprising a forward primer and a reverse primer, wherein said forward primer binds to a complement said PAX 5 coding sequence at a position 5′ to codon 183 defined by nucleotides 501-503 of SEQ ID NO: 4 and wherein said reverse primer binds to said PAX 5 coding sequence 3′ to said codon 183, and wherein said amplifying generates an amplicon comprising PAX 5 codon 183, and (2) determining in said amplicon the amino acid encoded by said codon 183, wherein if said codon 183 encodes an amino other than glycine, or the similar small hydrophobic amino acids; alanine or cysteine, said human subject has an elevated risk of developing pre-B cell ALL. In some embodiments, codon 183 encodes an amino acid other than glycine that is serine.

In some embodiments, the method comprising determining the amino acid encoded by codon 183 in the octapeptide domain of PAX5 comprises sequencing the amplicon. In some embodiments, the method comprising determining the amino acid encoded by said codon 183 comprises hybridizing a probe to said amplicon. In some embodiments, the method comprising determining the amino acid encoded by said codon 183 comprises digesting said amplicon with a restriction endonuclease that recognizes a restriction site comprising the sequence GGC (i.e. glycine) or AGC (i.e. serine) at codon 183. In some embodiments, the amplicon is between about 50 base pairs and about 1,000 base pairs in length. In some embodiments, the method comprising determining the amino acid encoded by said codon 183 comprising digesting said amplicon with a restriction endonuclease that recognizes a restriction site comprising the sequence GGC (i.e. glycine) or AGC (i.e. serine) at codon 183, further comprises subjecting said digested amplicon to acrylamide gel or capillary electrophoresis.

In some embodiments, the method comprising an amplifying step comprises a PCR reaction. In some embodiments, the PCR reaction is a digital PCR reaction. In some embodiments, the PCR reaction is a digital PCR for sequence-tagged sites (STS) on chromosome 9 reaction. In some embodiments, the PCR comprises specificity for exon 5 of PAX5.

In some embodiments, the method for identifying a human subject having an elevated risk of developing pre-B cell acute lymphoblastic leukemia (ALL) comprising amplifying a nucleic acid comprising a region of the PAX5 coding sequence in a tissue sample from a human subject, wherein said amplifying uses a primer set comprising a forward primer and a reverse primer, comprises a forward primer comprising the sequence 5′-GGGTCAGTCCTTCTCAGTGC-3′ (SEQ ID NO: 21) and the reverse primer comprising the sequence 5′-ACTCGCTCCTCTGCAGGTAA-3′ (SEQ ID NO: 22).

In some embodiments, the methods comprise obtaining a tissue sample that is a germline sample. In some embodiments, the germline sample is a blood, buccal, or prenatal germline sample. In some embodiments, the methods comprise obtaining a tissue sample from a human subject exhibiting, or at risk of exhibiting, familial ALL, sporadic ALL, or a chromosome 9p abnormality.

In some embodiments, a method for identifying a human subject having an elevated risk of developing pre-B cell acute lymphoblastic leukemia (ALL) further comprises screening a sample from the subject for a chromosome 9p cytogenetic abnormality; wherein detection of the 9p cytogenetic abnormality is indicative of the need for periodic monitoring and/or treatment of ALL in the subject. In some embodiments, screening a sample from the subject for a chromosome 9p abnormality comprises cytogenetic screening, molecular screening or hydridization of said sample. In some embodiments, screening a sample from the subject for a chromosome 9p abnormality comprises fluorescence in situ hybridization (FISH). In some embodiments, the cytogenetic screening comprises comparative genomic hybridization (CGH) single nucleotide polymorphism (SNP) microarray.

In some embodiments, the chromosome 9p cytogenetic abnormality is selected from i(9)/dic(9;v); i(9)(q10)/dic(9;v); complete or partial loss of 9p; or homozygous deletion of CDKN2A/CDKN2B; copy neutral loss of heterozygosity of the germline PAX5 variant allele caused by loss of the chromosome 9p containing the wild type PAX5 allele and somatic duplication of the chromosome 9p containing the germline PAX5 variant allele. In some embodiments, the method for screening a sample from the subject for a chromosome 9p cytogenetic abnormality; comprises screening a somatic sample selected from a bone marrow sample or a tumor sample.

In some embodiments, a method for identifying a human subject having an elevated risk of developing pre-B cell acute lymphoblastic leukemia (ALL) as provided herein further comprises screening for a chromosome 9p cytogenetic abnormality further comprises comparing percent of mutant allele to wild-type allele to provide a ratio.

In some embodiments, a method for identifying a human subject having an elevated risk of developing pre-B cell acute lymphoblastic leukemia (ALL) as provided herein further comprises molecular cytogenetic screening selected from digital PCR for sequence-tagged sites (STS) on chr9p; digital karyotyping; quantitative fluorescent-PCR for loss of 9p; or shot-gun sequencing of free DNA for increased ratio STS on chr9p.

In some embodiments, a method for identifying a human subject having an elevated risk of developing pre-B cell acute lymphoblastic leukemia (ALL) as provided herein, and comprising detection of a chromosome 9p cytogenetic abnormality, further comprises administering induction therapy to the subject. In some embodiments, induction therapy is administered. In some embodiments, the induction therapy comprises administering L-asparaginase, Daunorubicin hydrochloride, Corticosteroid, and/or Vincristine sulfate.

In some embodiments, a method for selecting a human embryo for implantation is provided, the method comprising screening a sample from a human embryo by a method for identifying a human subject having an elevated risk of developing pre-B cell acute lymphoblastic leukemia (ALL) as provided herein; wherein if said codon 183 encodes glycine, the embryo is selected for implantation.

In some embodiments, a method for prenatal screening is provided, the method comprising screening a sample from a human fetus by a method for identifying a human subject having an elevated risk of developing pre-B cell acute lymphoblastic leukemia (ALL) as provided herein.

In some embodiments, a kit is provided for determining the presence and/or quantifying in a human subject at least one of (a) at least one mutation in a PAX5 gene comprising at least c.547G>A (p.Gly183Ser), in the octapeptide domain of PAX5 from SEQ ID NO:1; (b) a mutant mRNA encoding p.Gly183Ser PAX5 polypeptide of SEQ ID NO: 2; or (c) mutant p.Gly183Ser PAX5 polypeptide, having at least 75% sequence homology with SEQ ID NO: 2. In some embodiments, the kit comprises a set of primers and/or probes for amplifying and/or sequencing PAX5 DNA. In some embodiments, the set of primers comprises forward primer 5′-GGGTCAGTCCTTCTCAGTGC-3′ (SEQ ID NO: 21) and reverse primer 5′-ACTCGCTCCTCTGCAGGTAA-3′ (SEQ ID NO: 22). In some embodiments, the kit comprises a set of primers and/or probes for amplifying and/or sequencing PAX5 cDNA. In some embodiments, the set of primers comprises forward primer 5′-GCCAGAGGATAGTGGAACTTG-3′ (SEQ ID NO: 37) and reverse primer 5′-GTGGTGAAGATGTCTGAGTAGTG-3′ (SEQ ID NO: 38). In some embodiments, the kit comprises an antibody specific for binding an epitope comprising p.Gly183Ser in an antigen comprising a PAX5 polypeptide, having at least 75% sequence homology with SEQ ID NO: 2.

In some embodiments, a method is provided for identifying a candidate therapeutic compound for a human patient having (at risk of developing) pre-B cell acute lymphoblastic leukemia (ALL), said method comprising: (a) obtaining a transformed eukaryotic host cell containing a PAX 5 gene coding sequence comprising a codon 183 defined by nucleotides 547-549 (AGC) of SEQ ID NO: 1; (b) contacting said transformed eukaryotic host cell in the presence of a compound suspected of being a cancer therapeutic; (c) growing said transformed eukaryotic host cell in the absence of said compound; (d) determining the rate of growth of said host cell in the presence of said compound and the rate of growth of said host cell in the absence of said compound, and (e) comparing the growth rate of said host cells, wherein a slower rate of growth of said host cell in the presence of said compound is indicative of a candidate therapeutic for the treatment of a human patient having (at risk of developing) pre-B cell acute lymphoblastic leukemia (ALL). In some embodiments, the candidate therapeutic compound is selected from a nucleic acid, protein, antibody, or small molecule. In some embodiments, the host cell is an ALL patient derived cell. In some embodiments, the rate of growth is determined by a ³H-thymidine incorporation assay, a colony forming assay, or growth in a severe combined immunodeficiency (SCID), non-obese diabetic (NOD)/SCID or NSG mouse model.

In some embodiments, an isolated DNA is provided comprising an altered PAX5 DNA comprising at least c.547G>A (p.Gly183Ser) in the octapeptide domain of PAX5 and at least 90% homology with the nucleotide sequence of SEQ ID NO: 1. In some embodiments, a replicative cloning vector is provided, comprising an isolated DNA comprising an altered PAX5 DNA comprising at least c.547G>A (p.Gly183Ser) in the octapeptide domain of PAX5 and at least 90% homology with the nucleotide sequence of SEQ ID NO: 1.

In some embodiments, an expression system is provided, comprising an isolated DNA comprising an altered PAX5 DNA comprising at least c.547G>A (p.Gly183Ser) in the octapeptide domain of PAX5 and at least 90% homology with the nucleotide sequence of SEQ ID NO: 1. In some embodiments, the expression system, comprising an isolated DNA comprising an altered PAX5 DNA comprising at least c.547G>A (p.Gly183Ser) in the octapeptide domain of PAX5 and at least 90% homology with the nucleotide sequence of SEQ ID NO: 1, is operably linked to suitable control sequences. In some embodiments, a host cell is provided transformed with an expression system, comprising an isolated DNA comprising an altered PAX5 DNA comprising at least c.547G>A (p.Gly183Ser) in the octapeptide domain of PAX5 and at least 90% homology with the nucleotide sequence of SEQ ID NO: 1, wherein the expression system is capable of expressing cDNA encoding PAX5 G183S.

In some embodiments, a method for identifying a human subject having an elevated risk of developing pre-B cell acute lymphoblastic leukemia (ALL) is provided, wherein said human subject exhibits a chromosome 9p abnormality, said method comprising (a) amplifying a nucleic acid comprising a region of the PAX5 coding sequence of SEQ ID NO: 1 in a tissue sample from said human subject, wherein said amplifying uses a primer set comprising a forward primer and a reverse primer, wherein said forward primer binds to a complement of said PAX 5 coding sequence at a position 5′ to codon 183 defined by nucleotides 547-549 of SEQ ID NO: 1 and wherein said reverse primer binds to said PAX 5 coding sequence 3′ to said codon 183, and wherein said amplifying generates an amplicon comprising PAX 5 codon 183, and (b) determining in said amplicon the amino acid encoded by said codon 183, wherein if said codon 183 encodes an amino other than glycine, or the similar small hydrophobic amino acids; alanine or cysteine, said human subject has an elevated risk of developing pre-B cell ALL. In some embodiments, codon 183 encodes an amino acid other than glycine that is serine.

In some embodiments, induction therapy is administered. In some embodiments, the induction therapy comprises administering L-asparaginase, Asparaginase Erwinia chrysanthem, Daunorubicin hydrochloride, Corticosteroid and/or Vincristine sulfate.

In some embodiments, methods are provided for identifying germline mutation carriers, wherein loss of the normal 9p is an early leukemia initiating event.

In some embodiments, methods are provided for screening mutation carriers in the risk period for the loss of the normal 9p in order to diagnose leukemia earlier to improve prognosis.

In some embodiments, methods are provided for screening carriers of chr 9p-, to determine the underlying germline mutation in PAX5. In some embodiments, methods are provided to screen leukemias that have 9p- to identify germline mutations and allow for cascade testing of other family members to determine carrier status.

In some embodiments, methods are provided for identification of germline mutation carriers of the p.G183S PAX5 mutation. Methods are provided for testing in familial ALL families, wherein if the p.G183S PAX5 mutation is identified, cascade carrier testing of relatives for risk-stratification is performed.

In some embodiments, methods are provided for preimplantation genetic diagnosis, following in vitro fertilization. In some embodiments, the methods and compositions provided herein are used for selecting an embryo for implantation, prenatal diagnosis of germline mutation status, prenatal diagnosis of ALL.

In some embodiments, methods are provided for screening/surveillance of germline mutation carriers for ALL, comprising testing in sporadic ALL with 9p-, wherein if the p.G183S PAX5 mutation is identified, then cascade carrier testing of relatives is performed for risk-stratification.

In some embodiments, methods are provided for germline variant testing in familial ALL families comprising genotyping for recurrent variant PAX5 c.547G>A, p.G183S.

In some embodiments, screening for carriers of a mutation is performed by full sequencing and deletion/duplication analysis of PAX5.

In some embodiments, germline variant testing is performed in sporadic ALL with somatic complete or partial loss of 9p secondary to cytogenetic abnormalities like i(9)(q10)/dic(9;v); or copy neutral loss of heterozygosity of the germline PAX5 variant allele caused by loss of the chromosome 9p containing the wild type PAX5 allele and somatic duplication of the chromosome 9p containing the germline PAX5 variant allele; or with somatic aberration of PAX5 due to focal or broad deletion; or copy neutral loss of heterozygosity of the germline PAX5 variant allele caused by loss of the chromosome 9p containing the wild type PAX5 allele and somatic duplication of the chromosome 9p containing the germline PAX5 variant allele.

In some embodiments, a two tiered method for germline mutation screening or carrier screening is provided. Genotyping for recurrent variant PAX5 c.547G>A, p.G183S is performed as Tier 1 of germline mutation screening or for carrier screening; and full sequencing and deletion/duplication analysis of PAX5 is performed as Tier 2 of germline mutation or carrier screening.

In some embodiments, a test kit is provided comprising reagents, and a sheet of instructions and/or software for determining the presence and/or amount of: a) at least one mutation in a PAX5 gene comprising PAX5 c.547G>A, p.G183S; b) mutant mRNA encoding PAX5 polypeptide, and/or c) the mutant polypeptide PAX5 p.G183S. In some embodiments, the kit for detecting mutant polypeptide PAX5 p.G183S comprises an antibody specific for the mutation.

In some embodiments, a kit is provided for determining a mutation in a PAX5 protein, said kit comprising an antibody that binds to mutant p.Gly183Ser PAX5 polypeptide, having at least 75% sequence identity with SEQ ID NO: 2; wherein the antibody does not bind to p.Gly183Gly PAX5 polypeptide having at least 75% sequence identity with SEQ ID NO: 2. In some embodiments, a kit is provided for determining a mutation in a PAX5 protein, said kit comprising an antibody that binds to mutant p.Gly183Ser PAX5 polypeptide, having at least 75%, 90%, 95%, or at least 99% sequence identity, with SEQ ID NO: 2; wherein the antibody does not bind to p.Gly183Gly PAX5 polypeptide having at least 75%, 90%, 95%, or at least 99% sequence identity with SEQ ID NO: 2.

In some embodiments, a method for surveillance of carriers for loss of wild type 9p is provided. In some embodiments, a method comprising screening for somatic loss of 9p (9p-) is provided. In some embodiments, determination of the ratio of mutant to wild type base of the G183 mutation is performed using digital PCR for exon 5. In some embodiments, Digital PCR is performed for the mutation to identify % of alternate versus wild type allele. In certain aspects this is performed in tandem with 9p-assessment to ensure loss of wild type allele and retention of the mutant.

In some embodiments, methods are provided for determining loss of chr 9p- in the subject, wherein a sample is obtained from the subject, and a method is performed for determining loss of chr 9p- in the subject selected from 1) digital PCR for STS on chr9p; 2) digital karyotyping utilizing serum or plasma; 3) quantitative fluorescent-PCR for loss of 9p; or 4) shot-gun sequencing of free DNA for increased ratio STS on chr9p. In some aspects, quantitative fluorescent-PCR is performed for loss of 9p, for use in prenatal testing, to calculate the ratios of STRs of a particular chromosome.

In some embodiments, a primer is provided for amplifying a nucleic acid comprising a region of PAX5, wherein the primer comprises a region of the sequence of SEQ ID NO: 37 or SEQ ID NO: 38, wherein the primer is from 8 to 200 nucleotides in length.

In some embodiments, a probe is provided for hybridizing a nucleic acid comprising a region of PAX5, wherein the probe comprises a region of the sequence of SEQ ID NO: 37 or SEQ ID NO: 38, wherein the probe is from 8 to 200 nucleotides in length, and wherein the probe is detectably labeled.

### BRIEF DESCRIPTION OF THE DRAWINGS

FIG. 1A shows Family 1 of Puerto Rican ancestry. The proband is denoted by an arrow. Exome sequencing was undertaken in germline DNA from all available affected (IV1, IV5, IV6, III5) and unaffected (IV9, III3, III4) individuals as well as the diagnostic leukemic sample from IV6. p.Gly183Ser variant status denoted by (+/−).

FIG. 1B shows Family 2 of African-American ancestry. The proband is denoted by an arrow. Exome sequencing was undertaken in diagnostic, remission and relapse leukemic samples from individuals III4, IV1, and IV2. p.Gly183Ser variant status denoted by (+/−).

FIG. 1C shows Chromosome 9 copy number heat map for SNP6.0 microarray data of germline and tumor samples from three members of Family 2. These data demonstrate the common feature of loss of 9p in the tumor specimens. Contiguous dark grey regions from centromere to chromosome 9p telomere (Chr 9p tel) indicate loss of 9p. Contiguous dark grey regions from centromere to chromosome 9q telomere (Chr 9q tel) indicate gain of 9q, G, germline; D, diagnosis; R, relapse sample.

FIG. 1D shows the haplotype flanking the p.Gly183Ser mutation. A five SNP haplotype rs7850825 to rs7020413 (Chr9:36.997-37.002 Mb) proximal to the mutation was concordant in both family 1 and family 2. However, the distal end flanking the mutation rs6476606 was discordant.

FIG. 2 shows Recurrent PAX5 mutations in ALL.

FIG. 2 A shows a gene schematic of PAX5 showing the exons (upper grey numbers), amino acid residues (lower grey numbers), protein domains (as denoted by legend) and position of the germline p.Gly183Ser variant (*) in relation to the somatic PAX5 mutations described in this study (n=13, arrows) and somatic mutations described previously in B-ALL^{1,2,20}. Primary leukemic samples with confirmed retention of the germline p.Gly183Ser variant denoted by the square shape (Family 1) and diamond shape (Family 2). In one case of i(9)/dic(9) ALL, both a heterozygous Va126Gly and a heterozygous Gln350fs mutation were observed, indicating polyclonality of the tumor.

FIG. 2 B shows conservation of the octapeptide domain in selected PAX family members PAX 2 (SEQ ID NO: 39), PAX3 (SEQ ID NO: 40), PAX5 (SEQ ID NO: 41), PAX 6 (SEQ ID NO: 42) and PAX 8 (SEQ ID NO: 43).

FIGS. 3A-D show attenuated transcriptional activity of PAX5 p.Gly183Ser. FIG. 3A shows transcriptional activity of PAX5 variants compared to wild-type using a Pax5-dependent reporter gene assay in 293T cells. Bars show mean (±s.e.m.) luciferase activity of six individual experiments with triplicate measurements (PAX5 p.Gly183Val and PAX5 d2-6 four experiments with triplicate measurements). Asterisks indicate significant difference calculated by Dunnett's test (P<0.0001). MIR, MSCV-IRES-mRFP empty vector.

FIG. 3B shows transcriptional activity of PAX5 variants using CD79A-dependent sIgM expression in the murine J558LμM plastocytoma cell line. Percentages indicate proportion of mRFP positive cells that show sIgM expression. Bars show mean (±s.e.m.) sIgM expression in two individual experiments with triplicate replicates each. Asterisks indicate significant difference calculated by Dunnett's test (P<0.0001). MIR, MSCV-IRES-mRFP empty vector.

FIG. 3C shows PAX5-dependent reporter gene assay of PAX5 wild-type and PAX5 p.Gly183Ser run in triplicate as above, with or without co-transfection of 0.05 μg of Grg4 as indicated. A PAX5 p.Tyr179Glu (Y179E) mutant that is deficient in binding to Grg4 and empty vector were used as controls. Asterisks indicate significant differences by two-tailed t-test (p<0.0001).

FIG. 3D shows a heatmap of PAX5 activated genes in mature B cells. Four samples from family 2 (diagnosis and relapse samples from individuals IV1 and IV2) show differential expression of PAX5 activated genes when compared to a group of 139 sporadic B-ALL cases. This indicates an effect of the pGly183Ser mutation on PAX5 function. Within the heatmap, medium grey indicates high expression, dark grey represents low expression. PAX5 mutation status is indicated by the rectangular boxes above the samples: dark grey indicates wild type PAX5, light grey indicates heterozygosity for a PAX5 mutation, and dark grey with a black dot indicates biallelic PAX5 mutations.

FIG. 4 shows position of germline PAX5 variant on chromosome 9, HG 19 position 37002702, exon 5 with a nucleotide sequence change C>T/G>A, resulting in amino acid change G183S. Germline PAX5 variant coding nucleotide sequence (SEQ ID NO: 1) and translation amino acid sequence (SEQ ID NO: 2) are also shown. Underlined sequence indicates alternate exons. Bold indicates amino acids encoded across a splice junction.

FIG. 5 shows regions of genomic nucleotide sequences for germline PAX5 variant for (+) strand (SEQ ID NO: 3) and (−) strand (SEQ ID NO: 4). The primer sequences are underlined.

FIG. 6 shows Table 2A with PAX5 primer sequences and sequencing primers for various exons 1A-10, labeled as SEQ ID NO: 5 to SEQ ID NO: 36; and Table 2B with PAX5 cDNA primers SEQ ID NO: 37 and SEQ ID NO: 38.

FIG. 7 shows somatic copy number aberrations in IV6, family 1. Affymetrix SNP 6.0 array on the diagnostic leukemic sample from individual IV6 from family 1.

FIG. 8 shows Principal Component Analysis of family 1 with 1000 Genomes reference populations. The first two principal components (EV1 and EV2) were plotted for Family 1 (ALL-1) in Red; together with reference populations from 1000 genomes such as CEU, CHB, ASW, PUR and YRI. Acronyms: Members from family 1 (ALL-1), HapMap individuals of African ancestry in Southwest USA (ASW), Utah residents with Northern and Western European ancestry from the CEPH collection (CEU), Han Chinese in Beijing, China (CHB), Puerto Rican in Puerto Rico (PUR), Yoruba in Ibadan, Nigeria (YRI). The data show the samples from Family 1 (ALL-1) clustering with the Puerto Rican (PUR) subset from 1000 Genomes.

FIG. 9 shows proper nuclear distribution of recombinant hPAX5. Hek293 cells were transiently transfected with WT or p.Gly183Ser human PAX5 as indicated. Cell lysates were lysed and a sucrose-density gradient was used to separate each total lysate (T) into its nuclear (N) and cytosolic (C) components. Fractions were resolved via SDS-PAGE, Western blotted, and probed with indicated antibodies to confirm nuclear localization of recombinant PAX5. WT and p.Gly183Ser PAX5 show primarily nuclear distribution (i) and even appear more nuclear fraction specific than the nuclear localization positive control, Splicing Factor-2 (SF2) (ii). Probing for β-actin to observe its predominant presence in the cytosolic fraction (iii) further confirms adequate sucrose gradient nuclear and cytosolic fraction separation.

FIG. 10 shows Principal component analysis of gene expression data from transduced J558LμM cells. Unsupervised Principal component analysis (PCA) of gene expression data from J558LμM cells transduced with empty vector (yellow, N=7), or constructs containing PAX5 wild type (green, N=7), PAX5 p.Gly183Ser (red, N=7), PAX5 p.Gly183Val (blue, N=7), PAX5 p.Pro80Arg (black, N=6) and PAX5 Dexon 2-6 (aqua, N=6). PCA was performed using 1,000 representative probe sets selected by k-means.

FIG. 11 shows Enrichment Score Curves for transduced J588LμM cells. Gene set enrichment analysis demonstrates a negative enrichment for genes upregulated during differentiation of murine B lymphoid progenitors from Hardy stage D to E in cells transduced with mutant PAX5 p.Gly183Ser (FIG. 11A), p.Gly183Val (FIG. 11B), p.Pro80Arg (FIG. 11C), D2-6 (FIG. 11D) or empty vector MIR (FIG. 11E) compared to cells transduced with wild type PAX5. This indicates a reduction of genes upregulated by mutant PAX5 as compared to wild type PAX5 and thus loss of function. The HARDYWTE_V_WTD_UP500 geneset includes the top 500 probesets upregulated in Hardy Panel E (mature B cells) compared with Hardy Panel D (Pre-B cells), identified using limma³b bv. NES: normalized enrichment score, FDR: false discovery rate.

FIG. 12 shows Principal component analysis of gene expression data. Unsupervised principal component analysis of gene expression data from 139 sporadic childhood B-progenitor ALL samples and 4 familial ALL samples (Diagnosis and relapse samples from individuals IV1 and IV2 of family 2) using 1,000 representative probe sets. light grey: Familial ALL(Family-2 samples), dark grey: ETV6-RUNX1-ALL, medium grey: ERG ALL, light medium grey BCR-ABL1-like ALL, pale grey: BCR-ABL1 ALL, black: Hypodiploid ALL, medium dark grey: Hyperdiploid ALL.

FIG. 13 shows Enrichment Score Curves for childhood B-progenitor ALL samples (a-b). GSEA was used to examine enrichment of genes differentially expressed in ETV6-RUNX1 PAX5 mutant ALL in the gene expression profile of PAX5 p.Gly183Ser mutant ALL (defined by FPKM analysis of mRNA-seq data). The gene expression profile of PAX5 p.Gly183Ser mutant ALL was defined by comparing familial ALL cases compared to PAX5 wild type sporadic ALL cases excluding ETV6-RUNX1 ALL to avoid a confounding effect of ETV6-RUNX1 status on GSEA results. The ETV6 PAX5_MUT_VS_PAX5_WT_FDR_20PCT_DOWN gene set includes the genes down regulated in ETV6-RUNX1 rearranged ALL with a PAX5 mutation versus ETV6-RUNX1 rearranged ALL with wild type PAX5 identified using limma with a maximum false discovery rate of 20%. Conversely, the ETV6_PAX5_MUT_VS_PAX5_WT_FDR_20PCT_UP gene set includes the genes up regulated in ETV6-RUNX1 rearranged ALL with a PAX5 mutation when compared to ETV6-RUNX1 rearranged ALL with wild type PAX5. (FIG. 13A) GSEA shows positive enrichment of genes down regulated in PAX5 mutant ETV6-RUNX1 rearranged ALL in the signature of PAX5 wild type B-ALL without ETV6-RUNX1 rearrangements compared to p.Gly183Ser mutated familial ALL and (FIG. 13B) a negative enrichment of genes upregulated in ETV6-RUNX1 rearranged ALL with mutant PAX5 in the signature of p.Gly183Ser mutated familial ALL compared to PAX5 wild type B-ALL without ETV6-RUNX1 rearrangements. These results support the notion that the p.Gly183Ser mutation and loss of wild type PAX5 in the familial cases result in loss of PAX5 function. (FIGS. 13C-E) GSEA shows an enrichment of genes activated in mature B cells (FIG. 13C)², genes activated in the transition from pro-B to mature B cells (FIG. 13D)², and a negative enrichment for genes repressed in the transition from pro-B to mature B cells (FIG. 13E)², in non-ETV6-RUNX1 rearranged B-ALL versus p.Gly183Ser mutated familial ALL, illustrating a loss of function for PAX5 p.Gly183Ser. (FIG. 13F) GSEA demonstrates a negative enrichment for Hardy Panel fraction E down-regulated genes (mature B cell stage)³ in sporadic non-ETV6-RUNX1 rearranged B-ALL versus PAX5 p.Gly183Ser mutated familial ALL samples, indicating a reduction of genes down regulated in familial ALL samples. NES: normalized enrichment score, FDR: false discovery rate.

FIG. 14A shows mouse expression array data from J558LμM transduced cells and Percentage overlap with the Revilla-i-Domingo et al. Table 9 data (FIG. 20) and the expression differences between wild type PAX5, empty vector (MIR) and the PAX5 mutants, p.Gly183Ser, p.Gly183Val, p.Pro80Arg, and d2-6.

FIG. 14B shows expression data from transduced J558LμM cells (x-axis) and normalized probe level intensity (RMA) (y-axis).

FIG. 14C shows percentage overlap with the Revilla-i-Domingo et al. Table 9 data (FIG. 20) and the expression differences between the familial ALL tumor samples (FAMALL) and non ETV-B-ALL wild-type for PAX5 (nonETVBALL.PAX5WT), other B-ALLs wild type for PAX5 (OtherBALL.PAX5WT), and all B-ALL cases including those with PAX5 mutations (OtherBALL).

FIG. 14D shows RNASeq data from human B ALL samples (x-axis) and transcript expression levels estimated as Fragments Per Kilobase of transcript per Million mapped reads (FPKM) (y-axis) for SCAND1 (left panel) and SH3BP2 (right panel).

FIG. 15 shows Table 3 with a clinical summary of cytogenetic findings in Family 1 and Family 2. Sex chromosomes, X and Y, are each denoted as N.

FIG. 16 shows Tables 4A and 4B with candidate germline variants in Families 1 and 2. Table 4A shows candidate germline variants in Family 1 predicted to affect protein function (indels, non-synonymous and splice site variants) selected based on two different models dependent on whether IV-9 was a non-carrier (Model 1) versus whether they were a carrier and non-penetrant for disease (Model 2). Table 4B shows candidate germline variants predicted to affect protein function (indels, nonsense, non-synonymous and splice site variants) shared between affected individuals in Family 2.

FIG. 17 shows Table 5 with germline copy number aberrations identified by SNP6.0 array in family 2.

FIG. 18 shows Table 6 with frequency of PAX5 ‘CCTAA’ haplotype in 1000 Genome populations; and Table 7 with somatic variants identified by exome sequencing the diagnostic leukemia sample in individual IV6, family 1.

FIG. 19A shows Table 8A with validated somatic SNVs and indels identified in three individuals from family 2.

FIG. 19B shows Table 8B with additional validated somatic SNVs and indels identified in three individuals from family 2.

FIG. 20 shows Table 9 with somatic copy number aberrations identified by SNP6.0 array in three individuals from family 2.

FIG. 21 shows Table 10 with validated somatic fusions identified by RNASeq in the leukemia samples from individuals from family 2. D indicated diagnosis sample, R represents relapse sample. FIG. 21 also shows Table 11 with gene expression analysis of J558 cells transduced with WT PAX5 and PAX5-pGly183Ser, Pro-B cell targets.

FIG. 22 shows Table 12 with gene expression analysis of J558 cells transduced with WT PAX5 and PAX5-pGly183Ser, Mature B cell targets. FIG. 22 also shows Table 13 with gene-expression analysis of PAX5 transduced J558 cells. Pax5 targets, Wildtype vs. Empty Vector.

### DETAILED DESCRIPTION OF THE INVENTION

B-cell precursor acute lymphoblastic leukemia (ALL) is the most common pediatric malignancy. There is a 2-4 fold increased risk of developing the disease in children of affected siblings⁴, and in occasional cases ALL is inherited as a Mendelian disorder⁵. PAX5, encoding the B-cell lineage transcription factor paired box 5, is somatically deleted, rearranged or otherwise mutated in approximately 30% of sporadic B-progenitor ALL cases^{1,3,6-9}. In PAX5-deficient mice, B-cell development is arrested at the pro-B-cell stage, and in vitro these cells can differentiate into other lymphoid and myeloid lineages¹⁰. PAX5 is also essential for maintaining the identity and function of mature B-cells¹¹, and its deletion in mature B cells results in dedifferentiation to pro-B cells and aggressive lymphomagenesis¹².

Somatic alterations of the lymphoid transcription factor gene PAX5 are a hallmark of B-progenitor acute lymphoblastic leukemia (B-ALL)¹⁻³, but inherited mutations of PAX5 have not previously been described. A novel heterozygous germline variant, c.547G>A (p.Gly183Ser), in the octapeptide domain of PAX5 is disclosed herein that was found to segregate with disease in two unrelated kindreds with autosomal dominant B-ALL. Leukemic cells from all patients in both families exhibited 9p deletion, with loss-of-heterozygosity and retention of the mutant PAX5 allele at 9p13. Two additional sporadic ALL cases with 9p loss harbored somatic PAX5 Gly183 substitutions. Functional and gene expression analysis of the PAX5 mutation demonstrated relatively reduced transcriptional activity. These data extend the role of PAX5 alterations in the pathogenesis of pre-B ALL, and implicate PAX5 in a novel syndrome of susceptibility to pre-B cell neoplasia.

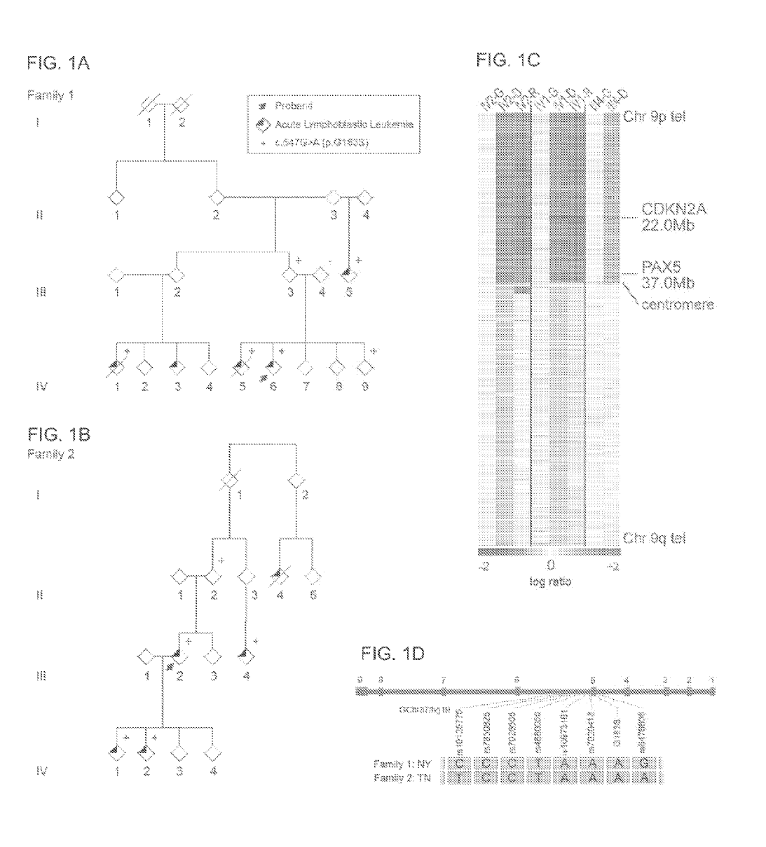

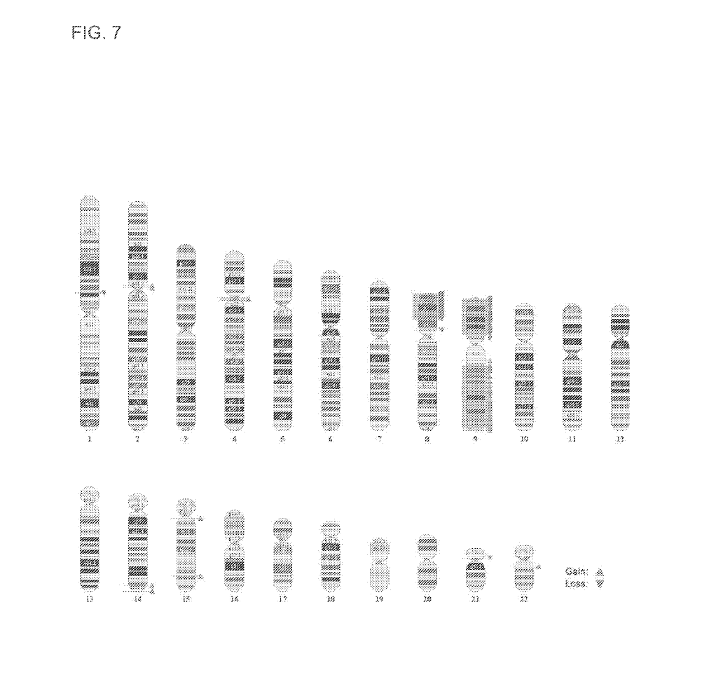

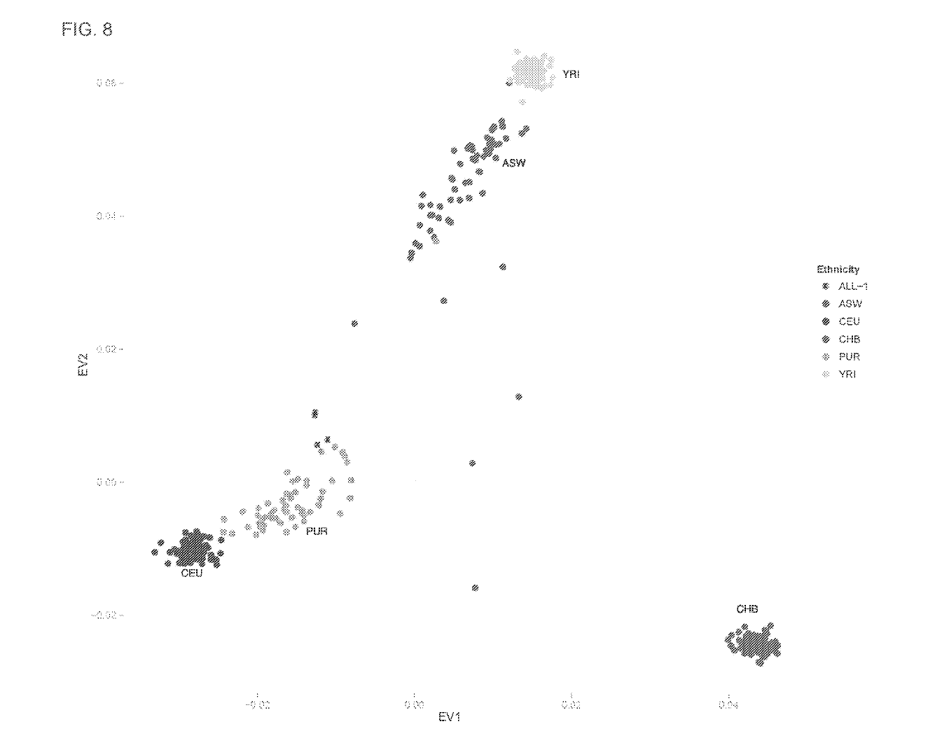

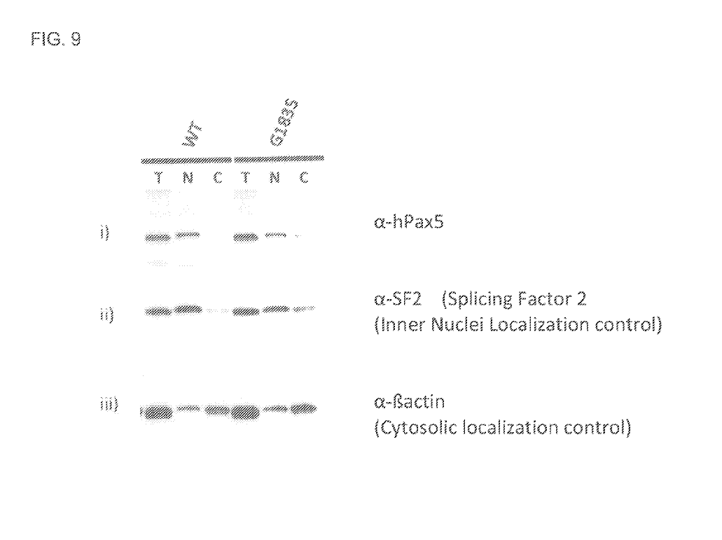

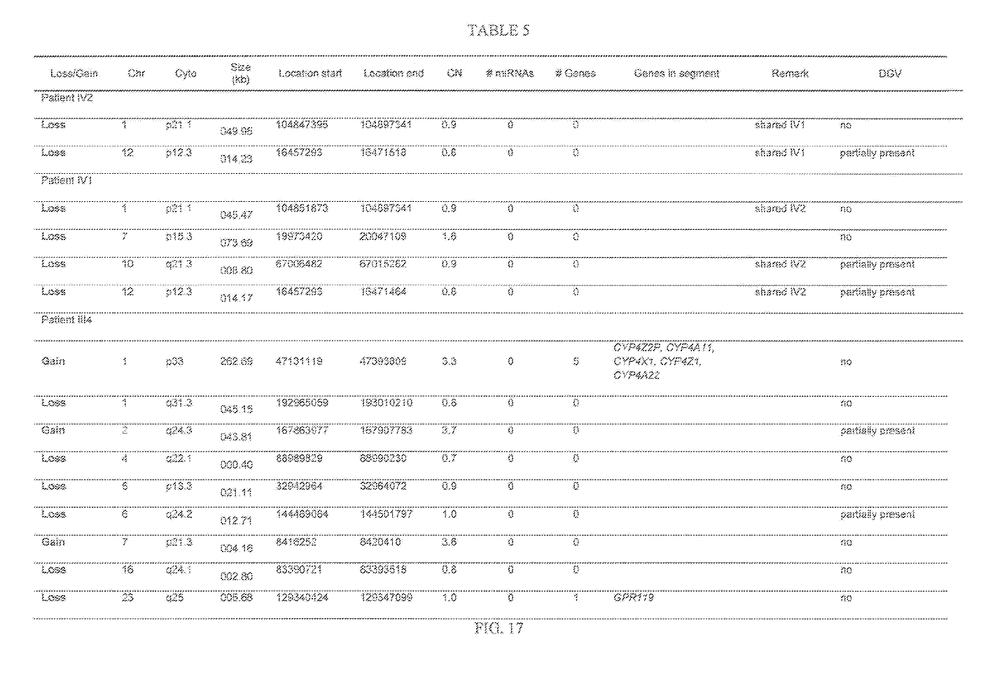

A heterozygous germline PAX5 variant, c.547G>A (NM_016734), p.Gly183Ser (NP_057953), was identified herein by exome sequencing in two families, one of Puerto Rican ancestry (family 1; FIG. 1A) and the other of African-American ancestry (family 2; FIG. 1B). This variant had not been previously described in public databases (Exome Variant Server, 1000 Genomes and dbSNP 137), or previous sequencing analyses of ALL and cancer genomes^{1,2,9}. All affected family members had B-cell precursor ALL and all available diagnostic and relapse leukemic samples from both families demonstrated loss of 9p through i(9)(q10) or dicentric chromosomes involving 9q, both of which resulted in loss of the wild-type PAX5 allele and retention of PAX5 p.Gly183Ser (FIG. 1C, FIG. 7 and Table 3, FIG. 15).

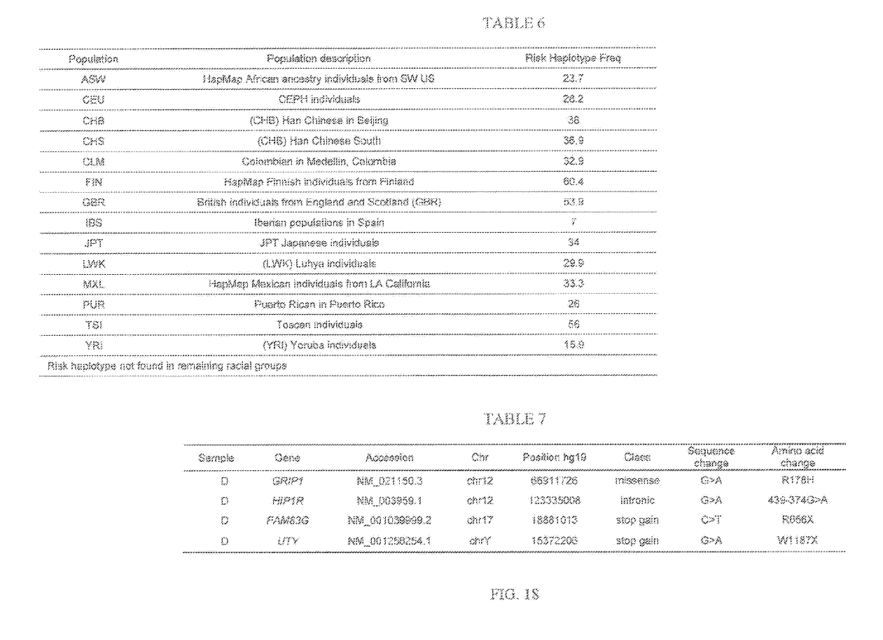

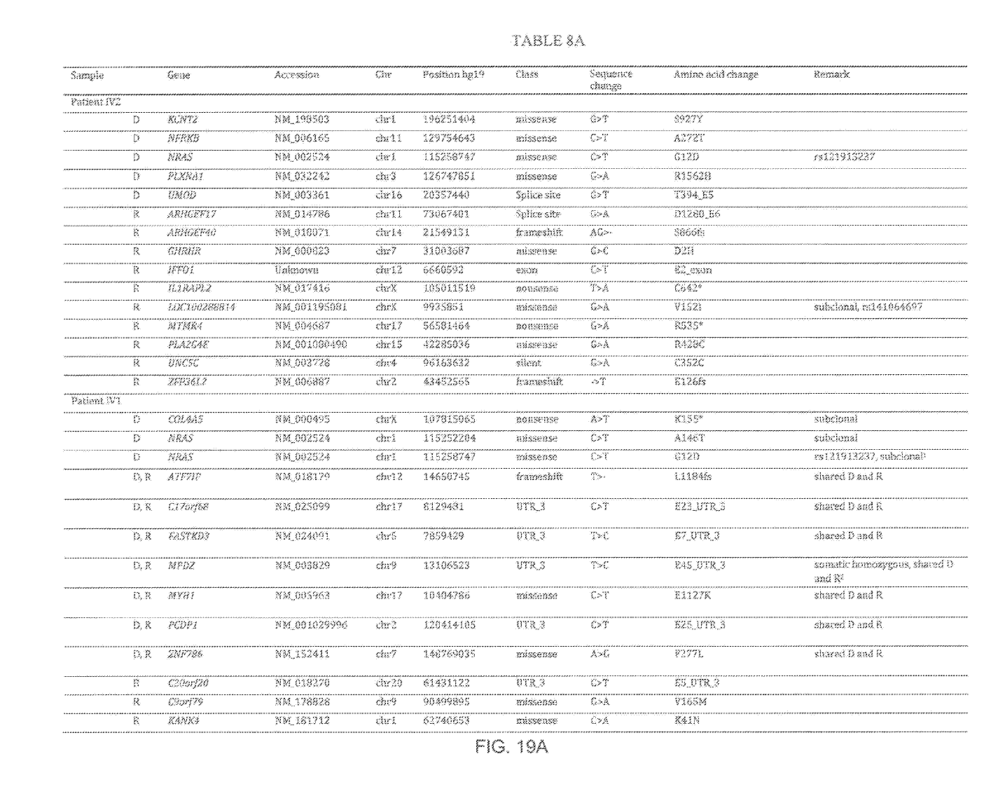

The germline PAX5 p.Gly183Ser mutation segregated with the leukemia in both kindreds, however, several unaffected obligate carriers (Family 1: II3, III2, III3 and Family 2: I1, I2, II2, II3) were also observed, suggesting incomplete penetrance. Unaffected mutation carriers and patients at the time of ALL diagnosis had normal immunoglobulin levels and no laboratory or clinical evidence of impaired B-cell function. Sanger sequencing of cDNA from peripheral blood of unaffected carriers indicated biallelic transcription of PAX5 (data not shown). The only mutated gene common to both families was PAX5 and no germline copy number aberrations were found to be shared between patients (Tables 4A and 4B, FIG. 16 and Table 5, FIG. 17).

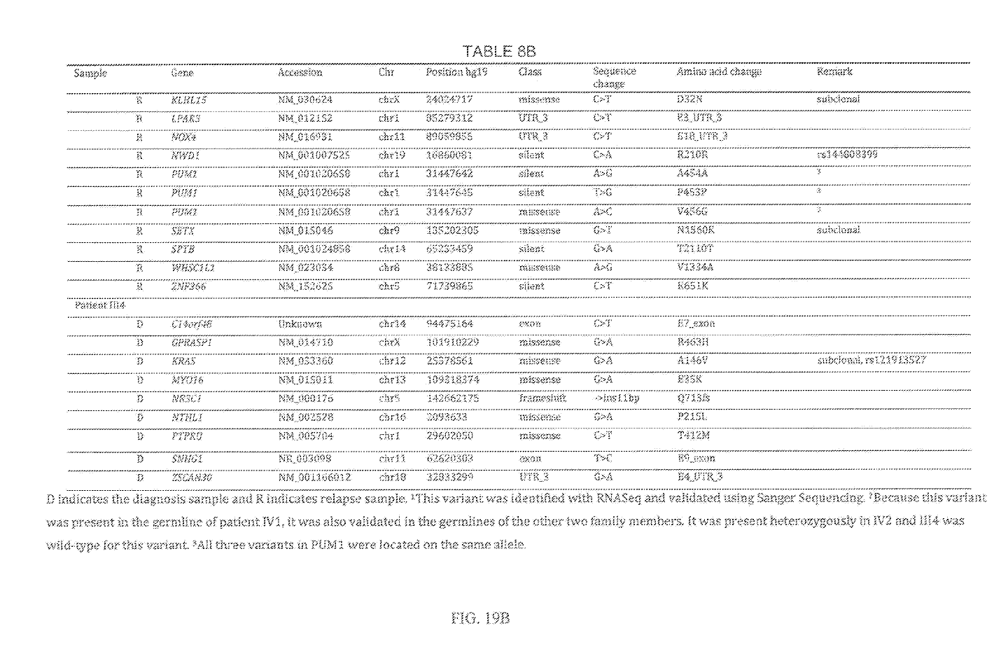

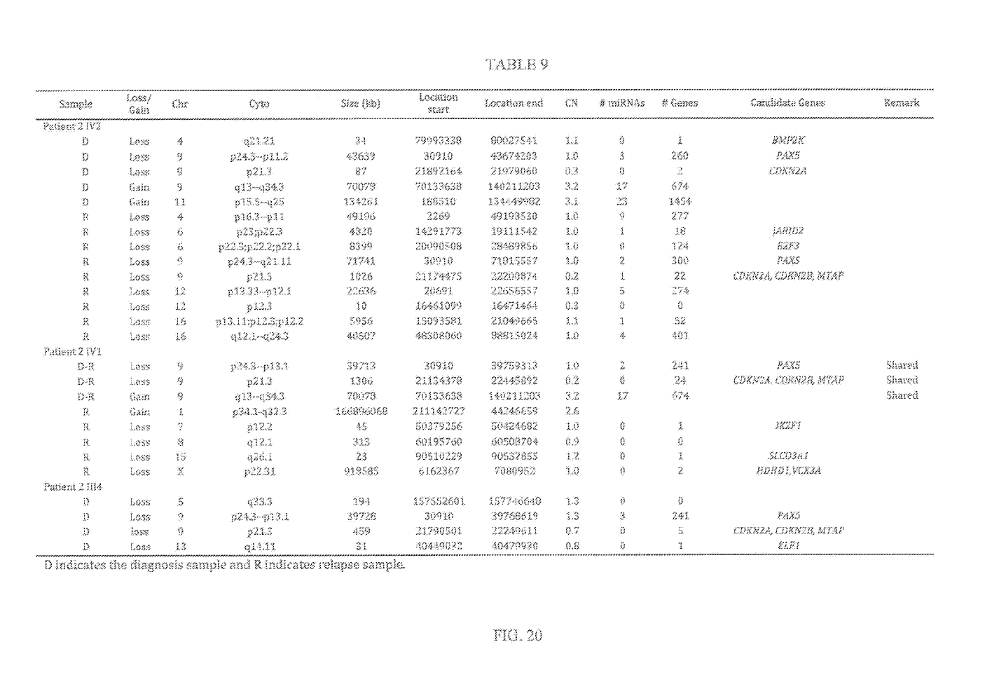

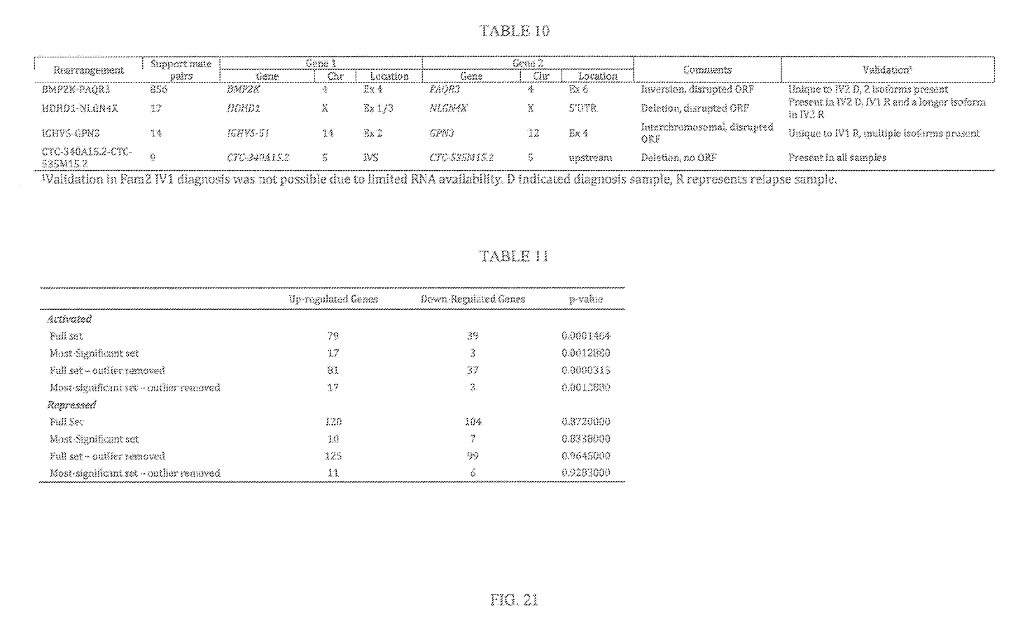

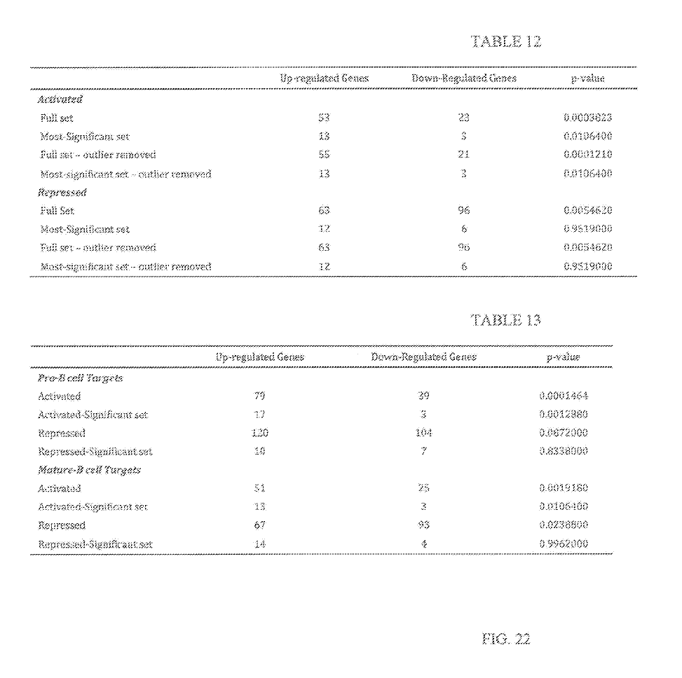

To determine whether p.Gly183Ser arose independently in each kindred or instead reflects common ancestry, the risk haplotypes of each family were compared. The families share a 4.7 kb haplotype, spanning five SNPs (FIG. 1D). The relatively small size of the shared haplotype and principal component analysis of genome-wide single nucleotide polymorphism genotype data (FIG. 8) together imply that the two families are not recently related and differ in ethnicity. Moreover, given the reduced fitness due to increased susceptibility for childhood ALL, it is unlikely that such a lethal mutation could be propagated over time. Because this haplotype is relatively frequent worldwide (Table 6, FIG. 18), it is likely that each family's mutation arose independently.

Genomic profiling of tumor samples demonstrated expression of the p.Gly183Ser mutant PAX5 in diagnostic and relapse tumor specimens from affected members of family 2, an average of 1 chimeric fusion and 9 non-silent sequence variants per case, and homozygous deletion of CDKN2A/CDKN2B in all cases due to loss of 9p and focal deletion of the second allele. Apart from loss of 9p, no other somatic sequence mutations or structural rearrangements were shared by the affected family members (Tables 3, 7-10)

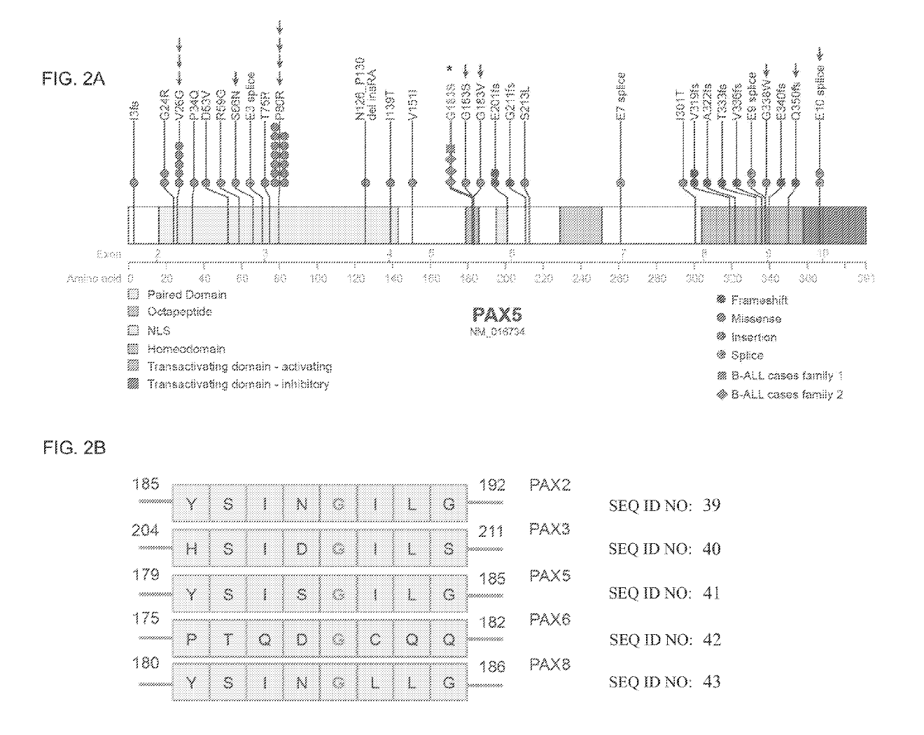

As somatic i(9)(q10)/dic(9;v) abnormalities were seen in all of the familial leukemias, we sequenced PAX5 in 44 additional sporadic pre-B ALL cases with i(9)(q10)/dic(9;v) aberrations to assess whether PAX5 mutations frequently co-occur with loss of 9p. Two leukemic samples revealed octapeptide mutations p.Gly183Ser, and p.Gly183Val, and in others previously reported variants including p.Pro80Arg and p.Va126Gly¹ were observed (Table 1, FIG. 2A). We examined the frequency of non-silent PAX5 somatic sequence mutations in a cohort of B-ALL cases with i(9)(q10)/dic(9;v) (n=28) and two cohorts of B-ALL without i(9)(q10)/dic(9;v) (n=183) and n=221.^{1,2}. We observed a significantly higher frequency of PAX5 mutations in the i(9)/dic(9) cohort (p=0.0001). No novel germline PAX5 mutations were detected in 39 families with a history of two or more cases of cancer including at least one hematological cancer, although one familial case of ALL harbored a dic(9;20)(p11;q11.1) and a somatic p.Pro80Arg variant (Table 1).

TABLE 1
PAX5 mutations found in familial and sporadic
i(9)(q10)/dic(9; v) pre-B cell ALL samples

| Inheritance | Patient | Mutation | Tumor status | Germline status |
|---|---|---|---|---|
| Family 1 | IV6 | c.547G > A = p.Gly183Ser | Homozygous | Heterozygous |
| Family 2 | III4 | c.547G > A = p.Gly183Ser | Homozygous | Heterozygous |
| Family 2 | IV1 D | c.547G > A = p.Gly183Ser | Homozygous | Heterozygous |
| Family 2 | IV1 R | c.547G > A = p.Gly183Ser | Homozygous | |
| Family 2 | IV2 D | c.547G > A = p.Gly183Ser | Homozygous | Heterozygous |
| Family 2 | IV2 R | c.547G > A = p.Gly183Ser | Homozygous | |
| Familial¹ | | c.239C > G = p.Pro80Arg | Homozygous | Wildtype |
| | | (tumor shows | | |
| | | dic(9; 20)(p11; q11.1)) | | |
| Sporadic | | c.77T > G = p.Val26Gly | Heterozygous | Wildtype |
| Sporadic | | c.77T > G = p.Val26Gly | Heterozygous | Wildtype |
| Sporadic | | c.77T > G = p.Val26Gly | Heterozygous | Wildtype |
| Sporadic | | c.197G > A = p.Ser66Asn | Homozygous | ND |
| Sporadic | | c.239C > G = p.Pro80Arg | Homozygous | Wildtype |
| Sporadic | | c.239C > G = p.Pro80Arg | Homozygous | Wildtype |
| Sporadic | | c.239C > G = p.Pro80Arg | Homozygous | Wildtype |
| Sporadic | | c.547G > A = p.Gly183Ser | Homozygous | ND |
| Sporadic | | c.548G > T = p.Gly183Val | Heterozygous | Wildtype |
| Sporadic | | c.1012G > T = p.Gly338Trp | Heterozygous | Wildtype |
| Sporadic | | c.1049- | Heterozygous | Wildtype |
| | | 1051delAGTinsGTCCG = | | |
| | | p.Gln350fs | | |
| Sporadic | | c.1100_1100 + 15 del16bp | heterozygous | ND |
| | | (IVS9 splice) | | |

¹Table 1. Familal case as reported in main text.
ND = Not Determinable.
Germline DNA either not tested or not available. PAX5 accession No: NM_016734

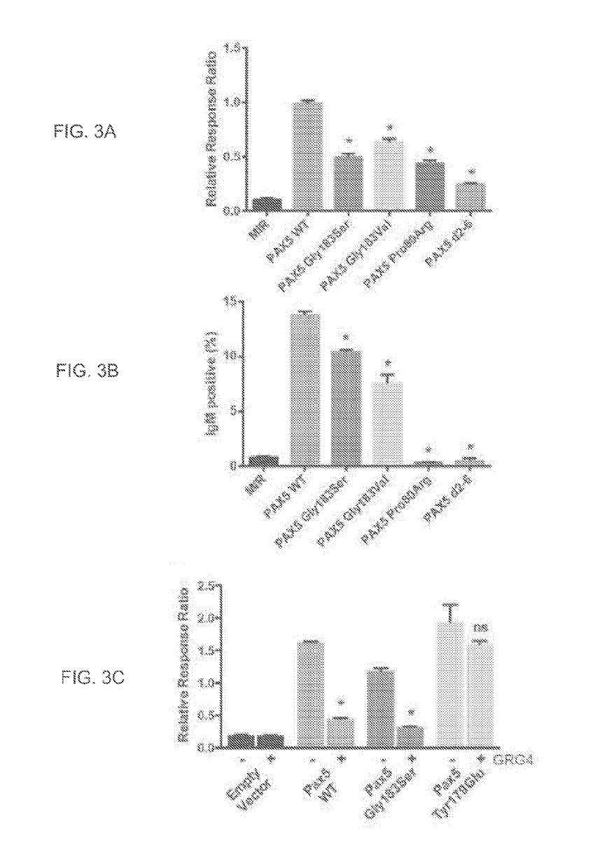

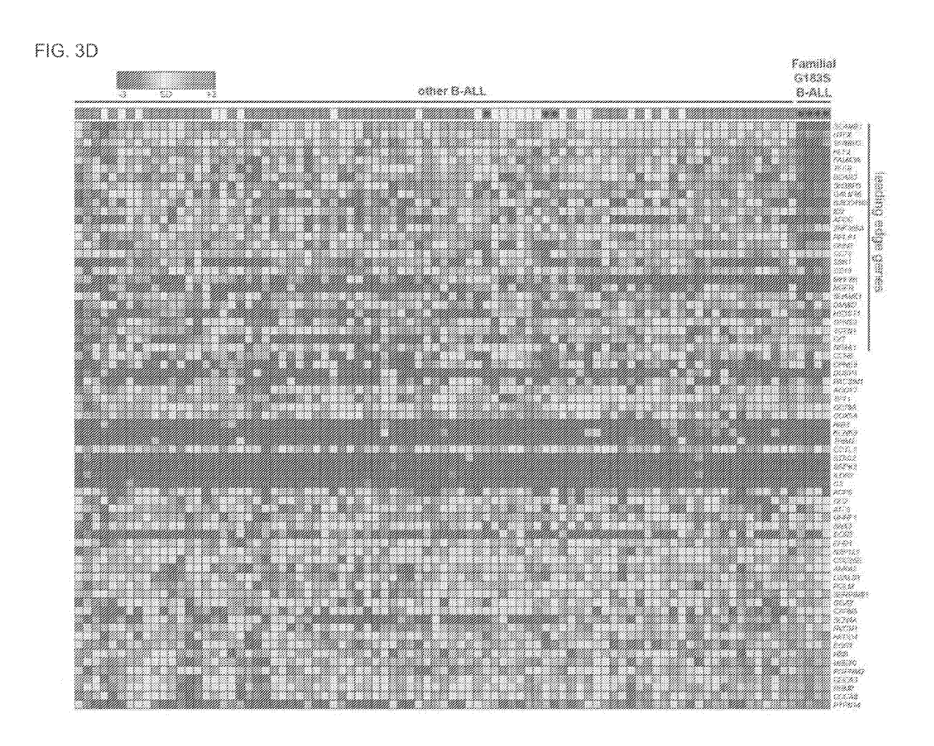

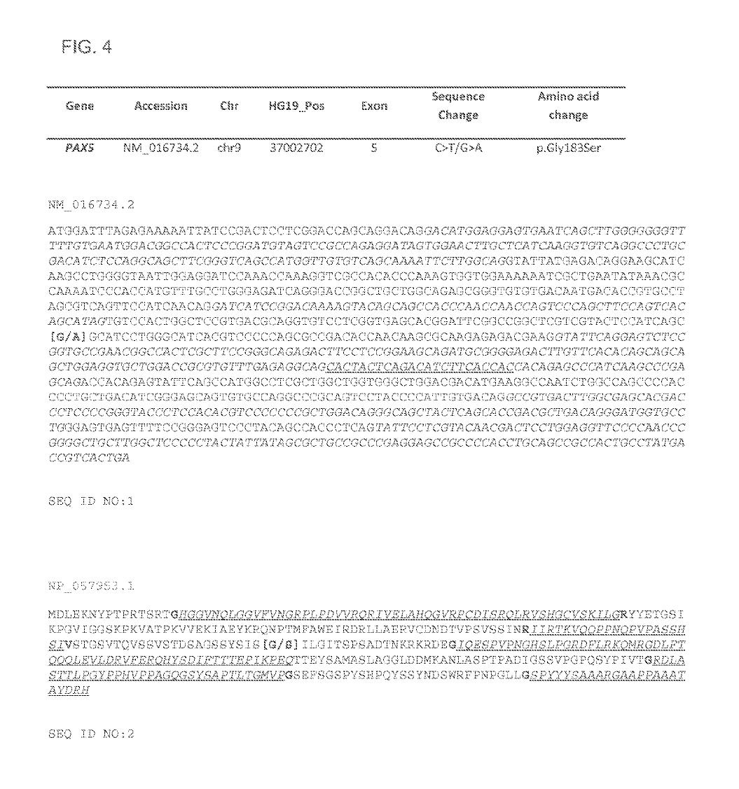

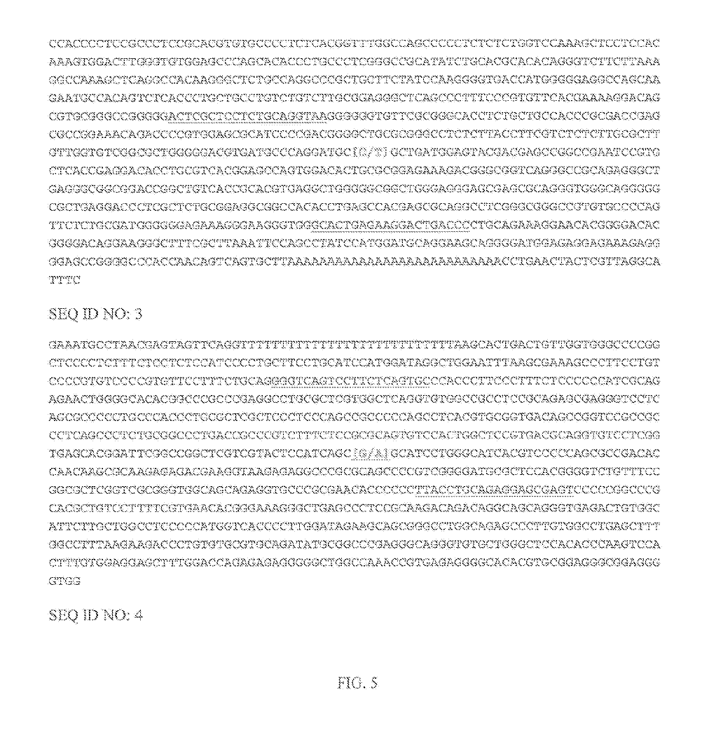

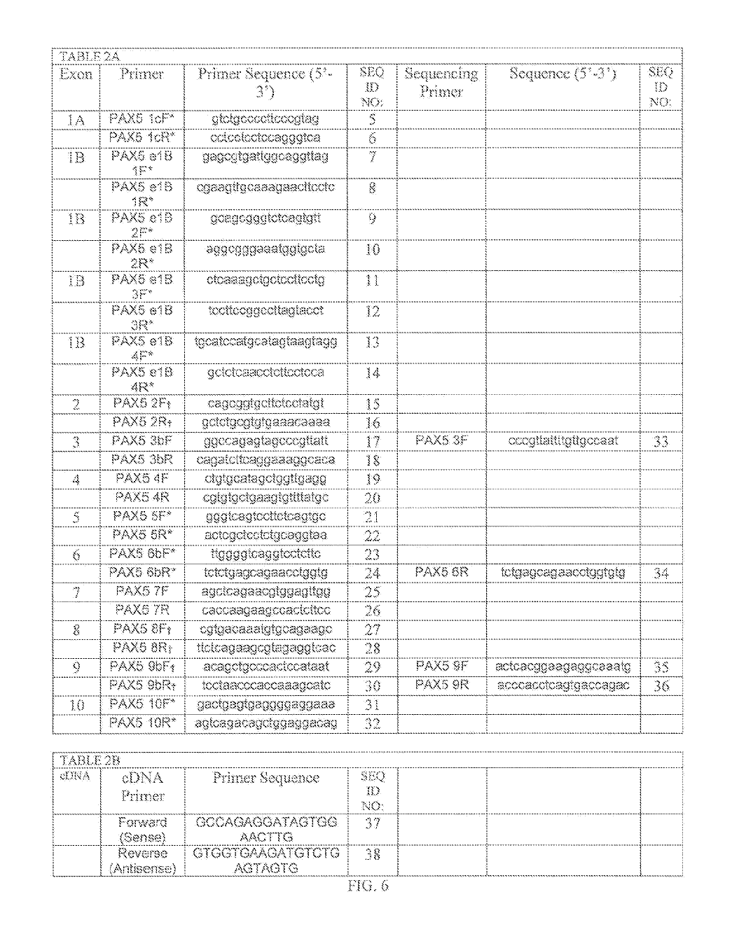

Previously identified PAX5 somatic mutations commonly result in significant reduction in transcriptional activation mediated by PAX5. Downstream targets of PAX5 include CD19 and CD79A (Igα or mb-1)¹³. The transactivating activity of wild type and mutant PAX5 alleles was examined herein using a PAX5-dependent reporter gene assay, containing copies of a high-affinity PAX5-binding site derived from the CD19 promoter¹⁴. Both p.Gly183Ser and Val mutations resulted in partial but significant reduction in transcriptional activation (P<0.0001 for both alleles, FIG. 3A). Additionally, there was no detectable differences in subcellular localization of wild type and p.Gly183Ser PAX5 (FIG. 9).

To study the effect of this mutation on CD79A expression, mutant and wild type PAX5 were expressed in J558LμM, a mouse plasmacytoma cell line that does not express PAX5 or CD79A. Enforced expression of PAX5 results in expression of CD79A and assembly of the surface immunoglobulin (sIgM) complex. The amount of sIgM expression may be used to assess the transcriptional activity of PAX5 alleles on the CD79A promoter¹. Both p.Gly183 alleles resulted in a significant reduction in sIgM expression compared to wild-type PAX5 (P<0.0001, FIG. 3B). These results suggest that the PAX5 G183 mutations result in partial loss of PAX5 activity.

The identified missense variant, p.Gly183Ser, is located at a conserved residue in the octapeptide (OP) domain of PAX5 that mediates interaction with Groucho transcriptional corepressors¹⁵ (FIG. 2B). Previous studies have shown that GRG4 (also known as TLE4 in humans) represses PAX5-dependent luciferase activity in cells expressing wild type PAX5 but not in cells expressing PAX5 octapeptide domain mutants¹⁵. We observed GRG4-mediated repression of the transcriptional activity of PAX5 wild-type and p.Gly183Ser (FIG. 3C), suggesting the effect of this mutation is not mediated by an altered interaction with GRG4.

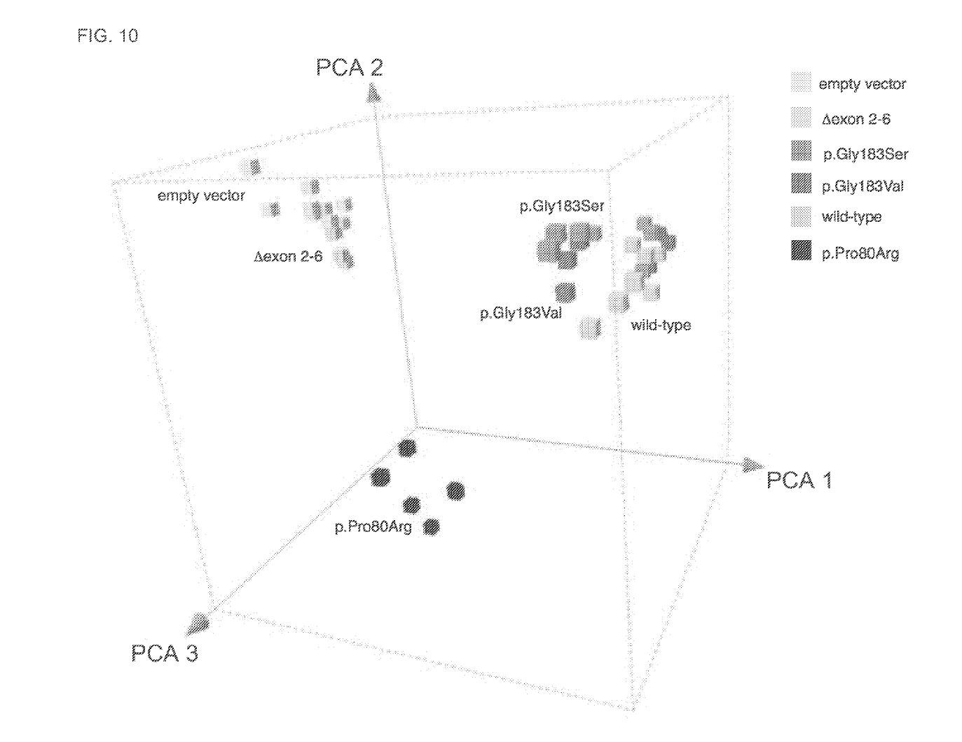

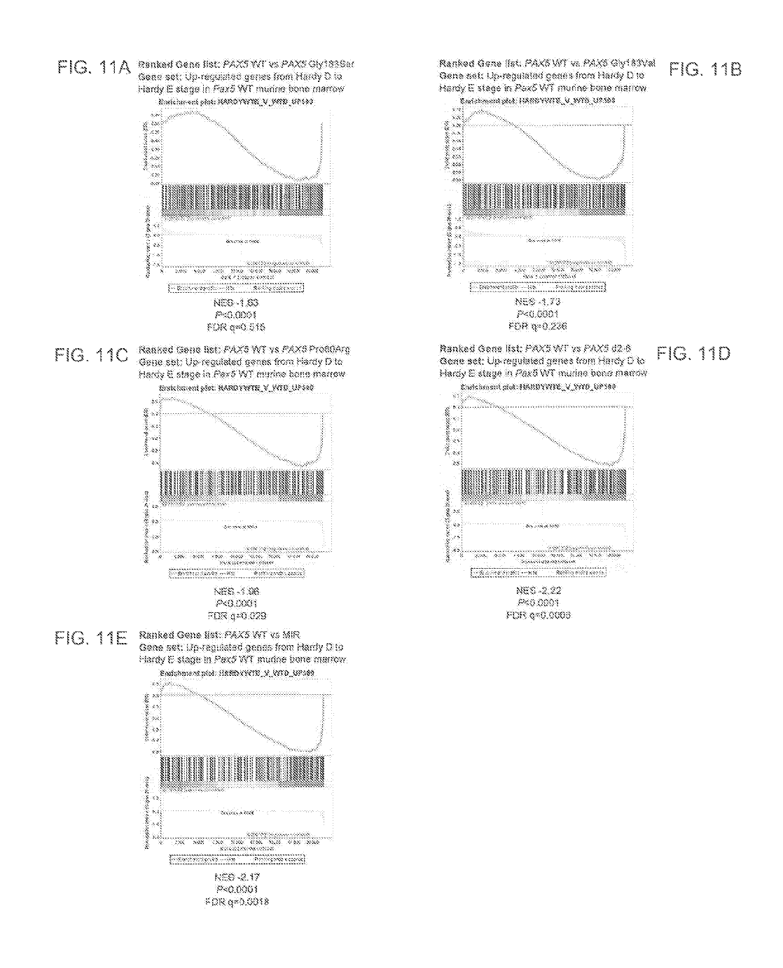

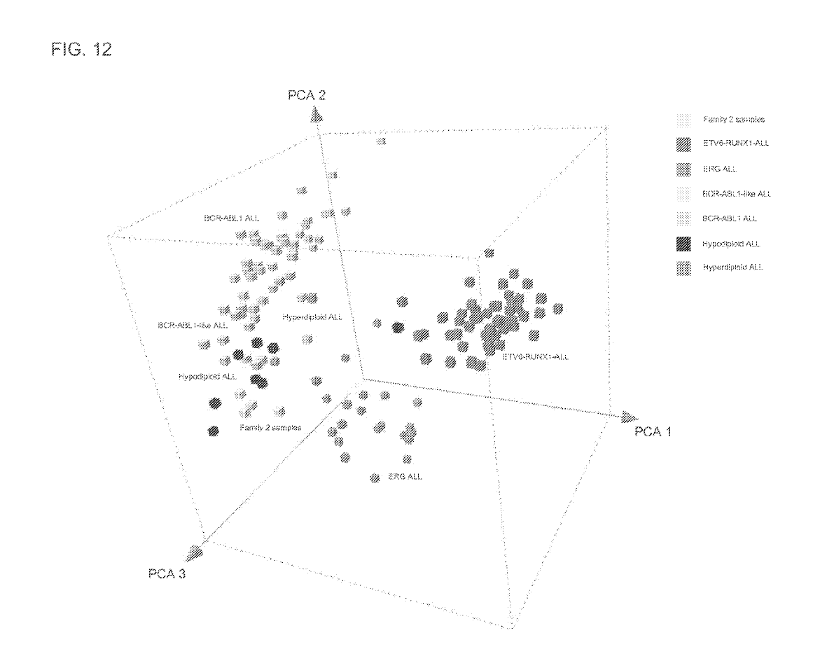

To further explore the effect of the variant on downstream targets, we performed genome-wide transcriptional profiling of J558LμM cells transduced with empty vector, PAX5 wild-type or mutant alleles (either all transduced cells marked by RFP expression, or the subset of cells expressing sIgM), and examined the expression of PAX5 activated and repressed genes previously defined in Pax5^{-/-} murine pro- and mature B cells¹⁶⁻¹⁹ and in human ETV6-RUNX1 B-ALL¹. Examining all PAX5 expressing cells, we observed profound deregulation of PAX5 activated and repressed genes in J558LμM cells expressing known loss of function (e.g. the common exon 2-6 deletion that results in a truncating frameshift PAX5 allele) or strongly hypomorphic alleles (PAX5 p.Pro80Arg) and less marked deregulation in p.Gly183Ser or Val expressing cells (P values for each mutant allele versus wild-type were all P<0.001 (FIGS. 10 and 11). Comparing sorted sIgM positive PAX5 p.Gly183Ser to PAX5 wild-type cells, we observed reduced expression of genes activated by PAX5 in pro-B and mature B-cells (P=1.4×10⁻⁴ and P=3.8×10⁻⁴ respectively, Table 11.

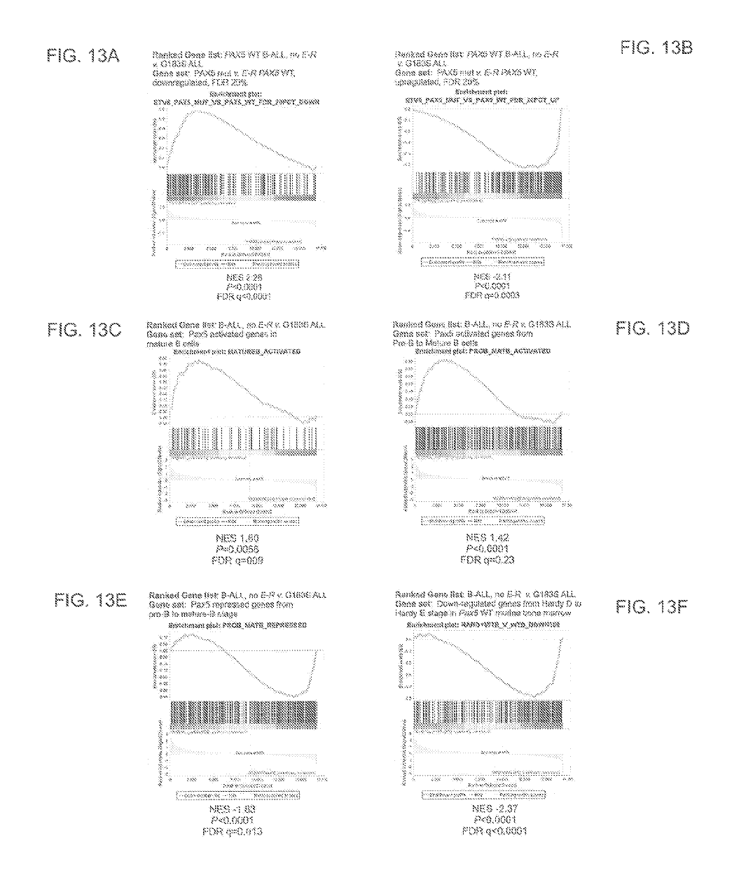

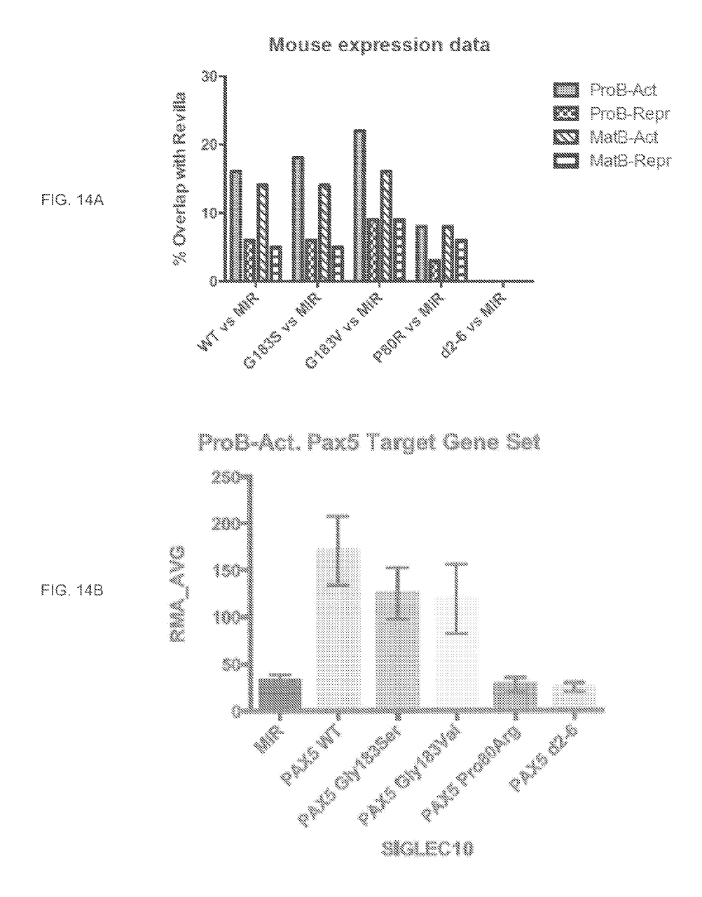

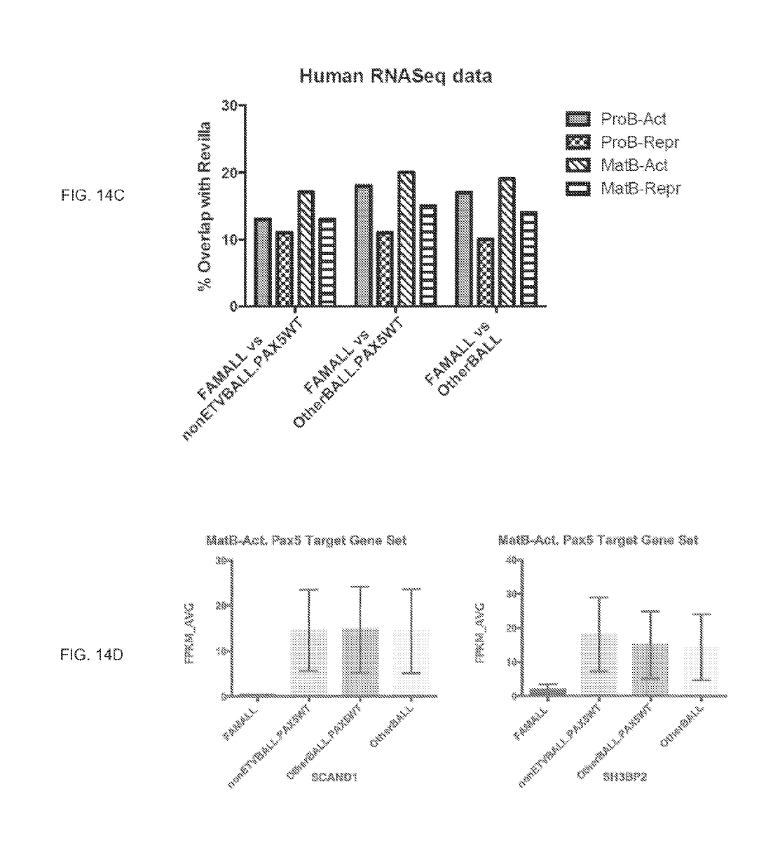

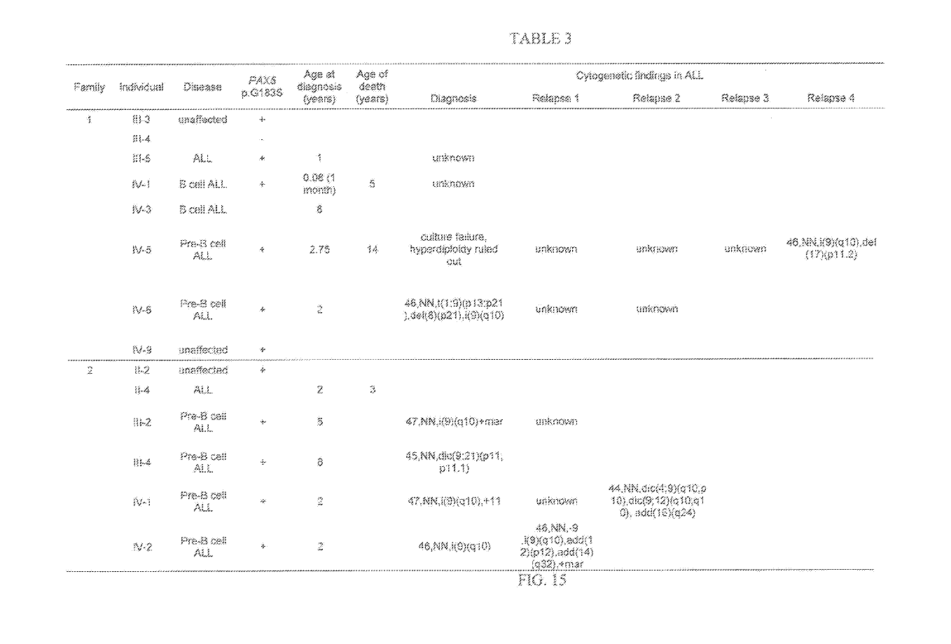

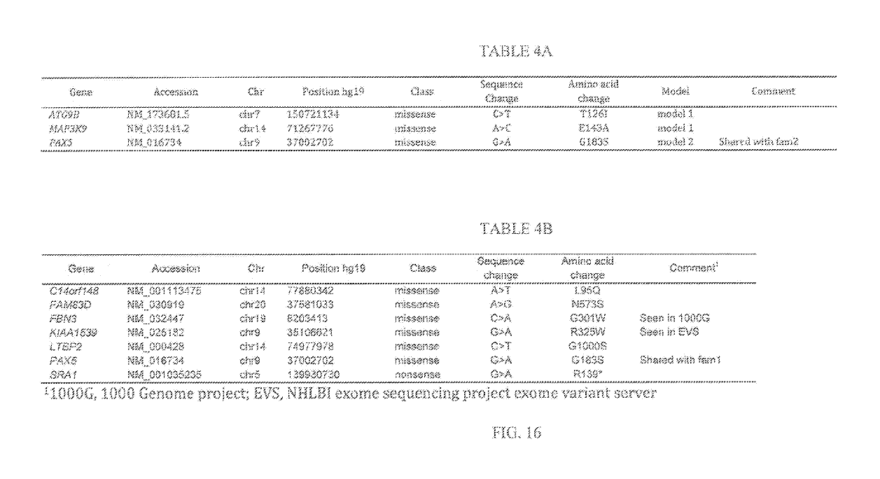

The transcriptional consequences of PAX5 p.Gly183Ser were next examined by performing transcriptome sequencing (mRNA-seq) of diagnosis and relapse samples obtained from two patients in kindred 2, and of 139 sporadic childhood B-progenitor ALL samples. We performed gene set enrichment analysis incorporating gene sets of PAX5-mutated ETV6-RUNX1 ALL (one third of which harbor focal PAX5 deletions)¹, PAX5 regulated genes in Pax5^{-/-} mice¹⁶⁻¹⁹ and genes regulated during murine B lymphoid development²⁰. As a limited set of genes are known to be regulated in both murine pro- and mature B cells, and as the overlap between mouse and human PAX5 regulated genes are unknown, we used all previously published PAX5 regulated genes, and genes regulated during murine B cell development¹⁶⁻²⁰ in an unbiased approach to explore the effects of the PAX5 p.Gly183 mutations on direct and indirect transcriptional targets of PAX5. This showed striking enrichment of genes deregulated in ETV6-RUNX1/PAX5-mutated ALL, PAX5 activated and repressed genes (including CD19, CD72 and CD79a), and genes regulated during murine B lymphoid development in the signature of familial PAX5 p.Gly183Ser versus sporadic B-ALL (FIG. 3D and FIGS. 13 and 13. We also analyzed the overlap of previously published data and the expression differences between the familial ALL tumor samples and other B-ALL cases stratified by PAX5 mutation status (FIG. 14). Together, these results suggest that PAX5 p.Gly183Ser results in attenuation of PAX5 function and deregulation of PAX5 target genes that is less severe than the previously reported p.Pro80Arg and D2-6 alleles that result in marked or complete loss of PAX5 activity.

The PAX5 deletions, translocations and sequence mutations identified as somatic events in B-ALL commonly affect the DNA-binding and transactivation domains and result in complete loss or marked attenuation of PAX5 transcriptional activity, but are rarely homozygous and not observed as inherited variants. Moreover, Pax5 loss promotes the development of B-ALL in experimental models which are commonly accompanied by the acquisition of second hits in Pax5²¹, indicating that profound loss of PAX5 activity is commonly a central event in leukemogenesis. In contrast, the inherited PAX5 p.Gly183Ser mutation results in modest attenuation of PAX5 activity, and is accompanied by somatic loss of the wild-type PAX5 allele due to 9p alterations during leukemogenesis. This model is also consistent with the finding of a significant association of somatic PAX5 hypomorphic mutations coincident with complete loss of the normal PAX5 allele in leukemic cells absent 9p. These observations suggest that a severe reduction in PAX5 activity is incompatible with normal B lymphoid development and is deleterious in carriers, but by contrast, the partial hypomorphic p.Gly183Ser allele is tolerated as a germline allele but additional genetic events further reducing PAX5 activity are required to establish the leukemic clone. The universal finding of deletion of wild-type PAX5 in all familial ALL cases, rather than the acquisition of additional hypomorphic PAX5 mutations, suggests that a complete loss of wild-type PAX5 activity is required for developmental arrest and loss of maturation. This is supported by our transcriptional profiling of J558LμM p.Gly183Ser cells and familial leukemias showing deregulation of PAX5 target gene expression that is significant but less marked than known loss-of-function mutations. The differences in transcriptional profiles of some target gene panels were not as robustly observed as in mouse model systems, presumably due to inherent germline and somatic genetic and epigenetic variability in human leukemias. In addition, ongoing studies will be of interest to fully characterize the functional consequences of PAX5 octapeptide domain mutations.

The findings disclosed herein have clinical implications with regard to options for pre-implantation genetic diagnosis, and the possible significance of somatic 9p alterations as a harbinger of a germline PAX5 mutation. The recent identification of germline TP53 mutations in familial ALL^{20,22} and the data presented here strongly implicating PAX5 mutations in a novel syndrome of inherited susceptibility to pre-B cell ALL, indicate that further sequencing of affected kindreds is required to define the full spectrum of germline variations contributing to ALL pathogenesis.

The mutation was identified in two families with multiple cases of childhood ALL, therefore the assumption is that this represents a high-penetrance leukemia susceptibility gene. There were however, obligate germline mutation carriers that were not affected with the disease. Without being bound by theory, the germline mutation causes there to be a greater than usual pool of premature B cells during the childhood-risk period that can allow for a greater chance of random loss of the 9p containing the normal PAX5 allele. Therefore, the particular cell that loses the normal PAX5 containing 9p would be more likely to transform to leukemia.

It is proposed that the 9p loss in the B ALL is the best indicator of a potential germline mutation in PAX5. This is based on the observation of 9p loss in all affected individuals of both families with the recurrent germline variant and the present finding of other previously reported PAX5 somatic sequence aberrations including two more events at the p.G183PAX5 residue in 44 sporadic cases of Pre BALL with i(9)/dic(9). Furthermore, review of previously studied B-ALL cohorts showed the frequency of non-silent PAX5 somatic sequence mutations in cases with i(9)(q10)/dic(9;v) (n=28) and two cohorts of B-ALL without i(9)(q10)/dic(9;v) (n=183) and n=221 to be significantly higher frequency in the i(9)/dic(9), cohort (p=0.0001). Taken together, these data support the notion that a percentage of PAX5 mutations in the germline would occur de novo and there would not necessarily be a family history, that supports the utility of sequencing sporadic ALL cases with somatic loss of 9p secondary to cytogenetic abnormalities like i(9)(q10)/dic(9;v) or with somatic aberration of PAX5 due to focal or broad deletion.

Although the degree of familial clustering of ALL is a rare syndrome, it is a recurrent mutation and the particular amino acid residue is also recurrently somatically mutated. The finding of the p.G183S PAX5 mutation in a sporadic ALL case with iso9q, although unconfirmed in the germline, provides basis to extend the role of germline PAX5 mutations beyond these rare families to sporadic ALL cases, which are common.

PAX5 is a critical gene in regulating B cell ontogeny. In some embodiments, germline variants were observed in other non-pediatric ALL malignancies, such as CLL, that could extend its role in predisposition to hematologic malignancies.

### EXAMPLES

### Accession Codes

Transcriptome and whole exome sequencing data and SNP microarray data have been deposited in the European Genome-phenome Archive (EGA) which is hosted by the European Bioinformatics Institute (EBI) under EGAS00001000447. Mouse Affymetrix gene expression data are deposited in the Gene Expression Omnibus (GEO) under accession code GSE45260 (http://www.ncbi.nlm.nih.gov/geo/query/acc.cgi?acc=GSE45260)

URLS.

dbSNP 137, http://www.ncbi.nlm.nih.gov/projects/SNP/; NHLBI exome sequencing project, http://evs.gs.washington.edu/EVS/; European Genome-phenome Archive, http://www.ebi.ac.uk/ega/. Public data portal for results from the St. Jude-Washington University Pediatric Cancer Genome Project, http://explore.pediatriccancergenomeproject.org/.

### Example 1

### Identification of a Recurrent Germline PAX5 Mutation and Susceptibility to Pre-B Cell Acute Lymphoblastic Leukemia

Patients and Samples.

Family 1 was ascertained from the Memorial Sloan-Kettering Cancer Center Clinical Genetics Service. Study subjects provided written informed consent as part of a study to define genomic causes of lymphoid malignancies and the study was approved by the local research ethics board. Family 2 from St Jude Children's Research Hospital was ascertained in accord with local IRB approval. To protect patient identity, pedigrees were anonymized by alterations that do not affect genetic analysis. FIGS. 1A-D show Familial Pre-B cell ALL associated with i(9)(q10) and dic(9;v) in Family 1 and Family 2 harboring a novel, recurrent germline p.Gly183Ser variant.

Family 1.

The proband in family 1 (IV6), denoted by an arrow in FIG. 1A, was a white Hispanic individual diagnosed with pre-B ALL at two years of age. Diagnostic bone marrow cytogenetic analysis revealed the abnormality 46,NN,t(1;9)(p13;p21),del(8)(p21),i(9)(q10) in 19 of 20 cells examined (FIG. 7, and Table 3, FIG. 15).

The sex chromosomes, X and Y, each are denoted as N. The disease course was characterized by two relapses five years apart, following which, an allogeneic hematopoietic stem cell transplant (HSCT) from an unrelated donor was undertaken. The proband remains in clinical remission six years from time of transplantation. The proband's sibling (IV5) was diagnosed with pre-B ALL at two years and nine months of age. The disease course was characterized by two central nervous system (CNS) relapses and a third relapse in the peripheral blood four years later. Cytogenetic analysis of the bone marrow during the third relapse revealed 46,NN,i(9)(q10),del(17)(p11.2) and homozygous loss of CDKN2A/B in the majority of cells examined. Limited clinical history is available for the parent's 26 year old half-sibling (III5), who was diagnosed with ALL at one year of age and the first cousin of the proband (IV1), who was diagnosed with B-cell ALL at one month of age and passed away at five years of age and another 13 year old cousin (IV3) diagnosed with B-cell ALL at eight years of age. The unaffected (obligate) carriers did not have any medical history consistent with signs or symptoms of impaired B-cell function. Analysis of PBMCs and serum from two unaffected carriers (III3 and IV9) were consistent with normal B-cell differentiation. In each case, flow cytometry showed most of the lymphocytes were T-cells. Within the B cell compartment, there was normal maturation as evidenced by expression of CD20 and CD22 but lack of expression of immature markers CD10, CD34 or TDT. Furthermore, there was no evidence of clonal excess. Additionally, serum electrophoresis and immunofixation electrophoresis of the samples were normal.

Family 2.

The proband in family 2, denoted by an arrow in FIG. 1B, is An African-American individual (III2) was diagnosed with ALL and CNS involvement at the age of 5 years. Leukemic blasts were of pre-B immunophenotype and cytogenetic studies demonstrated 47,NN,i(9)(q10)+mar. Following successful treatment of both the initial disease and a CNS relapse, clinical remission is maintained. Their child (IV1) was diagnosed with ALL at the age of 2 years; leukemic blasts had a B-progenitor immunophenotype, DNA index of 1.0 and a cytogenetic abnormality, 47,NN,i(9)(q10),+11. Disease course was characterized by a CNS relapse followed by a bone marrow relapse 2 years after completing salvage therapy. Cytogenetic study of the leukemic blasts at relapse revealed 44,NN,dic(4;9)(q10;p10), dic(9;12)(q10;q10),add(16)(q24). Following intensive re-induction therapy, the individual received an allogeneic HSCT and remains in clinical remission. A 2 year old sibling (IV2) was also diagnosed with ALL; leukemic cells had a transitional B-progenitor immunophenotype, DNA index of 1.0 and a cytogenetic abnormality: 46,NN,i(9)(q10). The sibling experienced a bone marrow relapse shortly after completing therapy. Cytogenetic study of the recurrent leukemia cells demonstrated 46,NN,-9,i(9)(q10),add(12)(p12),add(14)(q32),+mar. Following allogeneic hematopoietic stem cell transplant, clinical remission was achieved. The proband's cousin (III4) was diagnosed with ALL at 8 years of age. The leukemic cells had a pre-B immunophenotype, DNA index of 1.0 and a cytogenetic abnormality, 45,NN,dic(9;21)(p11;p11.1). The cousin responded well to induction and maintenance therapy and remains in continuous clinical remission. The proband's parent had a first cousin (II4) who was diagnosed with ALL at two years of age. Immunophenotypic and cytogenetic studies were not performed during this era of leukemia therapy. This individual responded well to induction and maintenance therapy, however, experienced a CNS relapse while on therapy and did not survive.

The unaffected carriers did not have any medical history consistent with signs or symptoms of impaired B-cell function. Analysis of PBMCs in one unaffected carrier was consistent with normal B cell differentiation.

Mutation Identification.

Analysis of the whole exome sequencing data from all available affected (IV1, IV5, IV6, III5) and unaffected (IV9, III3, III4) individuals of family 1 was carried out based on a presumed autosomal dominant mode of inheritance with incomplete penetrance. Variants occurring in family 1 were filtered to remove those seen in other in-house MSKCC exomes. Further filtering was undertaken to exclude those seen in public databases including 1000 genomes and the NHBLI exome variant server. Candidate variants were selected based on two different models dependent on whether IV9 was a non-carrier (Model 1) versus whether this individual was a carrier and non-penetrant for disease (Model 2). Filtering for only coding variants considered to effect protein function (indels, nonsynonymous and splice site variants) revealed 3 novel variants (two in Model 1 and one in Model 2), shared by all four affected individuals and the obligate carrier parent in family 1 (Table 4). The most compelling candidate was a heterozygous missense variant, c.547G>A (p.Gly183Ser), in the paired box protein encoding gene, PAX5. In silico analysis of this amino acid substitution using PolyPhen2 predicted it to be damaging (score 1.0). The missense variant segregated with disease in all affected individuals (IV1, IV5, IV6, III5) and was also present in the unaffected family members (III3, IV9), compatible with the observed autosomal dominant mode of inheritance with incomplete penetrance. For Family 2, germline exome sequencing was undertaken in three members of Family 2 (IV1, IV2 and III4) as previously described′ (Table 4). Seven variants (four novel) were found to segregate with affected individuals, but the only mutated gene shared between the two families was the c.547G>A (p.Gly183Ser) variant.

Exome Sequencing.

One microgram of germline DNA from peripheral leukocytes of affected individuals in remission and unaffected family members, was used for whole exome capture using the Agilent SureSelect 38 Mb or 50 Mb and paired-end sequencing with the Illumina HiSeq 2000²³. Family 1 exome data was analyzed using BWA²⁴ to align fastq files and generate BAM files, and GATK^{23,25} was used for variant calling. SNP cluster and proximity to indel, proportion of aligned reads at a site with mapping quality zero were used for filtering variants. Variant quality score recalibrated (VQSR) data was then processed using SNPEff program for functional annotation. Samples from Family 2 underwent variant analysis, as previously described²⁰. Downstream analysis consisted of filtering out low quality variant calls and those already reported in public databases. The downstream processing of sequence data, variant annotation and filtering strategy based on a presumed autosomal dominant mode of inheritance with incomplete penetrance was investigated.

Principal Component Analysis

From the exome-sequenced samples, single nucleotide variants seen at a frequency above 5% in the dbSNP were selected for principal component analysis. These data were then combined together with 1000 genomes SNP data. The SNPs were pruned on pairwise linkage disequilibrium within a 50 kb window. The data was transformed to calculate eigenvectors and eigenvalues for each sample and the first two principal components were plotted.

SNP Array Genotyping.

SNP array genotyping was performed using Affymetrix SNP 6.0 microarrays on the diagnostic leukemic sample from individual IV6 from family 1, and germline DNA from unaffected individuals III3, III4 and IV9 and analyzed using the Genotyping Console (Affymetrix). SNP 6.0 arrays were also performed for diagnostic leukemia and remission samples from individuals IV1, IV2 and III4 from family 2, as well as on the relapse samples from IV1 and IV2, and data was analyzed by optimal reference normalization²⁶ and circular binary segmentation^{27,28} as previously described²⁹ using R and dChip³⁰. Haplotype analysis was conducted using germline samples from III3, III4, IV9 and the diagnostic leukemic sample from IV6 from family 1 and the diagnostic and remission samples from IV1, IV2 and III4 from family 2.

In view of the cytogenetic abnormalities in each of the leukemic samples resulting in monosomy 9p, for which Sanger sequencing of the p.Gly183Ser variant demonstrated loss-of-heterozygosity with retention of the mutant allele, we were able to biologically-phase the SNP risk-haplotype containing the mutant allele. Beagle phased haplotypes from the 1000 Genomes were analyzed for the 5 SNP shared haplotype and the frequencies were estimated amongst the populations in HapMap.

Haplotype Analysis.

The biologically-phased SNP risk-haplotype derived from the leukemic sample from IV6, family 1, defined the risk haplotype in family 1 as a 4 MB region containing 1654 SNPs surrounding PAX5. This haplotype was validated in the germline diploid genotypes from the other members of the family. The germline risk-haplotype of family 2 was also inferred using the tumor genotype and this revealed the same finding of a biologically-phased risk haplotype surrounding the mutation in ALL specimens with loss of 9p from three individuals. Comparison of the two risk-haplotypes from each family demonstrated a shared five SNP haplotype rs7850825 to rs7020413 (Chr9:36.997-37.002 Mb) proximal to the mutation. The mutation is flanked by common SNPs rs7020413 and rs6476606, where the two families were discordant for rs6476606 (FIG. 1D). As the frequencies of the haplotypes in the Hapmap populations are relatively common (Table 6, FIG. 18), the fitness of this mutation, which causes a highly-penetrant fatal childhood-onset disease, would be predicted to be extremely low, our findings suggest that these mutations have likely arisen recently as two independent events, consistent with p.Gly183Ser being a recurrent mutation.

Tumor Analysis

Exome Sequencing.

The data from individual IV6 from family 1 was manually reviewed for the putative somatic variant calls to exclude those present at low frequencies in the BAM files and those with annotated rs IDs in combination with poor coverage in the germline. This resulted in four somatic SNVs (Table 7, FIG. 18). Somatic aberrations found by exome sequencing of diagnostic, remission and relapse samples from three individuals (III4, IV1, and IV2) from family 2 were validated using MiSeq (Illumina) and Sanger sequencing. Validated somatic SNVs and indels are presented in Table 8. Seven mutations present in the predominant clone of the diagnostic specimen of individual IV1 were also identified in the relapse specimen.

Copy Number Analysis.

Results of SNP array profiling in individual IV6 from family 1 were concordant with the proband's initial cytogenetic diagnosis of 46,NN,t(1;9)(p13;p21),del(8)(p21),i(9)(q10) (FIG. 7). Furthermore, there was homozygous loss at the CDKN2A/B locus at 9p21.3. A homozygous deletion of CDKN2A/B was also seen by FISH analysis in the relapse sample from individual IV5 from family 1. SNP array profiling was performed on the diagnostic and remission samples from individuals IV1, IV2 and III4 and relapse samples from IV1 and IV2 of family 2. Tumor aberrations included the common loss of 9p and also homozygous loss at the CDKN2A/CDKN2B locus at 9p21.3 in all specimens analyzed (FIG. 1C, Table 8). No germline copy number aberrations were shared between all three affected individuals were detected (Table 5, FIG. 17).

PAX5 Sequencing.

We used Sanger sequencing (primers available on request) on the entire ORF of PAX5 in sporadic ALL characterized by i(9)/dic(9;v) (n=44) and 31 cases of familial cancer, and reviewed the coding regions of PAX5 in an additional 8 families that had been exome-sequenced, or B-ALL cases that had been Sanger sequenced (n=87 treatment resistant adult-onset ALLs) as part of other studies. Cases were acquired from St. Jude Children's Research Hospital, Memphis Tenn. (n=34 i(9)/dic(9;v) and 28 familial cases), Memorial Sloan-Kettering Cancer Center/Columbia University, New York N.Y. (n=2 i(9)/dic(9;v) and n=87 treatment resistant adult-onset ALLs), Radboud University Nijmegen Medical Centre, the Netherlands (n=6 i(9)/dic(9;v)), Texas Children's Cancer Center and Human Genome Sequencing Center, Houston, Tex. (7 familial cases), Children's Cancer Institute Australia for Medical Research, Australia (n=2 i(9)/dic(9;v) and 3 familial cases), and from the Huntsman Cancer Institute/Primary Children's Medical Center, Salt Lake City, Utah (1 familial case).

DNA Constructs.

The CD19-luciferase construct used for the PAX5-dependent reporter gene assay, contains copies of a high-affinity PAX5-binding site (derived from the CD19 promoter)¹⁴ and was a kind gift from Dr. Meinrad Busslinger (IMP, Vienna, Austria). The pFLAG-CMV2-Grg4 construct was a kind gift from Dr. Gregory Dressler (University of Michigan, Ann Arbor, Mich.)³¹. The p.Gly183Ser and p.Gly183Val mutations were introduced into the pSG5 PAX5 WT, MSCV-IRES-mRFP-PAX5WT and pMSCV-PuroIRESGFP-PAX5WT vectors by site-directed mutagenesis (Quickchange, Agilent). For retroviral expression, the PAX5 WT and other mutant cDNAs were sub-cloned as a XhoI/EcoRI fragment into MSCV-Puro-IRES-GFP (MSCV-PIG) or MSCV-IRES-RFP vectors.

Cells and Antibodies.

Hek293 (ATCC CRL-1573) and Hek293T (ATCC CRL-11268) cells were maintained in Iscoves Modified Dulbecco' medium supplemented with 10% fetal calf serum and streptomycin. Parental J558 cells (ATCC TIB-6) were grown in Dulbecco's Modified Eagle's Medium with 10% horse serum³². J558LμM cells have been generated from a sub-line (J558L) that had lost heavy chain expression by infection with a cDNA of the membrane-bound heavy chain isoform³³ and were grown in RPMI 1640 media (Invitrogen, Carlbad, Calif.) supplemented with 10% fetal bovine serum (Hyclone, Logan, Utah), 2 mM L-glutamine (Invitrogen), 50 mg/ml gentamicin (Invitrogen), 0.3 μg/ml Xanthine (Sigma, St Louis, Mo.), and 1m/ml mycophenolic acid (Sigma) as previously described^{1,34}. Both lines (parental J558 and J558LμM) do not normally express surface IgM (sIgM) since they lack expression of CD79A³⁵, but partial expression of CD79A can be induced by exogenous expression of PAX5, leading to the upregulation of sIgM¹³. Retroviral supernatants were produced by transient transfection of Phoenix Eco cells with MSCV-PIG-PAX5 constructs, and used to infect J558 cells by spinoculation in the presence of 4 μg/ml Polybrene. Rabbit monoclonal anti-PAX5 (ab109443) and mouse monoclonal anti-Flag (ab18230) were purchased from Abcam. Mouse monoclonal anti-TLE4 (Grg4) (sc-365406) and anti-b-Actin (sc-1615) were purchased from Santa Cruz Biotechnology. Mouse monoclonal Anti-SF2 was purchased from Zymed (32-4500). Anti-IgM antibodies conjugated to R-phycoerythrin (PE) (553409) or Allophycocyanin (APC) (550676) were obtained from BD Pharmingen (BD Biosciences, San Jose, Calif.).

Subcellular Fractionation.

Protein expression and subcellular localization of the PAX5 wild-type and PAX5 p.Gly183Ser proteins were examined by sucrose-density gradient separated lysates from transiently transfected Hek293 cells. The sucrose gradient nuclei separation was adapted from Nuclei PurePrep Isolation Kit (Sigma, St Louis, Mo.). CF buffer (10 mM Tris-HCl, 1 mM MgCl2, 1 mM DTT, 10 uM PMSF) and 1.8M Sucrose Solution (Sigma) were used to create density layers for resolved separation by ultra-centrifugation. Fractions were then subjected to SDS-PAGE gel electrophoresis and immunoblotting with various antibodies to confirm adequate separation of nuclear and cytosolic fractions and determine localization of recombinant PAX5. Results are shown in FIG. 9.

Luciferase Assays.

293T cells were transfected with MIR/MSCV-PIG^{WT} or MIR/MSCV-PIG^{mutant}, along with luc-CD19 and pRL-TK Renilla luciferase plasmid DNA (Promega, Madison, Wis.) using Fugene 6 (Roche Diagnostics, Alameda, Calif.). For GRG4 repression assays, 500 ng of either the MSCV-PIG empty vector, MSCV-PIG PAX5 WT, MSCV-PIG PAX5 Gly183Ser or MSCV-PIG PAX5 Tyr179Glu mutant, 2 μg luc-CD19 construct and 0.1 μg pRL-TK Renilla luciferase plasmid were cotransfected with or without 50 ng GRG4 cDNA in pFLAG-CMV2 into HEK293T cells using X-tremeGENE HP DNA Transfection Reagent (Roche Diagnostics, Alameda, Calif.). Forty-eight hours post-transfection, cell lysis and measurement of firefly and Renilla luciferase activity was performed using the Dual-Luciferase® Reporter Assay System (Promega) according to the manufacturer's instructions. All transfections were performed in triplicate in at least two independent experiments. The firefly luciferase activity was normalized according to corresponding Renilla luciferase activity and reported as mean relative luciferase units (RLU)±s.e.m.

Flow Cytometric Analysis.

J558LμM cells transduced with MIR PAX5 vectors, or cells selected with puromycin after transduction with pMSCV-PIG vectors were analyzed for RFP (MIR) or GFP (pMSCV-PIG) and sIgM expression after staining with phycoerythrin- or allophycocyanin-conjugated anti-IgM antibodies (BD Pharmingen) using LSRII or Fortessa flow cytometers (Becton Dickinson, San Diego, Calif.).

Gene Expression Profiling.

The RFP positive fractions of J558LμM cells transduced with empty MIR vector and PAX5 wild type, 42-6, p.Pro80Arg, p.Gly183Ser and p.Gly183Val (N>6 replicate transductions) were flow sorted, expanded, purity checked and mRNA was extracted from 5-10×10⁶ cells using TRIzol (Invitrogen). mRNA was quantitated by spectrophotometry and integrity was assessed using an 2200 Tapestation (Agilent, Santa Clara, Calif.). Expression of wild-type and mutant PAX5 alleles was verified by RT-PCR and sequencing and immunoblotting. Gene expression profiling was performed using Mouse 430v2 PM arrays (Affymetrix, Santa Clara, Calif.) as previously described²⁰. Statistical analyses, principal component analysis and unsupervised hierarchical clustering were performed using R 2.15.2³⁶, Bioconductor 2.6³⁷ and Spotfire Decision Site 9.1.1 (Tibco) and Partek Genomics Suite version (Partek, St Louis, Mo.). Data was normalized import using the RMA algorithm³⁸. To adjust batch effects introduced by isolation and plate batches, the probe set signals were further corrected with the ComBat³⁹, which applies an empirical Bayes framework for adjusting data for batch effects. Probe sets not passing the background signal (twice the average signal on the control probes with different GC content) across all samples were excluded for differential expression analysis, where limma⁴⁰ with estimation of false-discovery rate (FDR)⁴¹. For Gene Set Enrichment Analysis (GSEA)⁴², we used gene sets obtained from the Molecular Signatures Database v3.0, Hardy Fraction (GSE38463)²⁰, and previous PAX5 studies^{13,14,17-19.} Gene sets with less than 10 or greater 500 genes were excluded, and significantly enriched gene sets after 1000 permutations at a FDR of <0.25 are reported. P-values were calculated using ANOVA with Dunnett's post hoc comparing each mutant allele to wild-type. We also analyzed the overlap with the Revilla-i-Domingo et al. data and the expression differences between wild type PAX5, empty vector (MIR) and the PAX5 mutants, p.Gly183Ser, p.Gly183Val p.Pro80Arg, and d2-6. For the analysis of the sIgM expressing subset, J558 cells were infected with either wild-type PAX5 (n=3), the p.Gly183Ser mutant (n=4), or an MSCV-PIG empty vector control (n=3) were stained with IgM-PE antibody and sorted for GFP/IgM(PE) double-positive cells (only single GFP⁺ cells in the case of the empty vector infected cells) on a FACSAriaII cell sorter (BD Biosciences, San Jose, Calif.). Total mRNA was extracted using TRIzol (Invitrogen) according to manufacturer's instruction, and gene-expression profiling was performed using Affymetrix GeneCHIP Mouse Gene 1.0 ST arrays. All of the data analysis was performed using the Partek Genomics Suite 6.5 (6.11.0207) (Partek, St. Louis, Mo.). The data was normalized upon import using RMA, and then an ANOVA analysis was performed to determine any differences among the conditions. One mutant sample was removed from the analysis based on its appearance as an outlier during principal component analysis. The genes used for analysis were constrained to those genes previously identified as being Pax5 targets in murine pro-B cells¹⁸. Genes were classified as being either activated or repressed by Pax5 (n=122; n=237, respectively). Revilla-i-Domingo also identified the most significant subsets of the activated or repressed Pax5 targets (n=20; n=21, respectively). P-values were calculated using the pbinom function in R 2.12.1.

Transcriptome Sequencing.

Transcriptome sequencing was performed on diagnosis and relapse samples obtained from two patients in kindred 2, and on 139 sporadic childhood B-progenitor ALL samples as described previously^{1,2,20}. The 139 sporadic childhood B-progenitor ALL samples included ETV6-RUNX1 (N=54), alteration of ERG (N=22), hyperdiploidy with greater than 50 chromosomes (N=1), hypodiploidy (N=8), BCR-ABL1 (N=27) and BCR-ABL1-like ALL (N=27). PAX5 deletion and mutation status was derived from whole exome/genome sequencing of all cases.

RNA-Seq Library Construction.

Total RNA was extracted using Trizol (Invitrogen, NY). Total RNA quality and quantity were assessed on Agilent RNA6000 Chip (Agilent, CA) and Qubit (Invitrogen, NY). Standard RNA-Seq was prepared from 1 μg total RNA following Illumina RNA-Seq protocols including DNase treatment and Phenol purification, PolyA+ RNA selection by using Oligo-dT beads, cDNA conversion, fragmentation by Covaris Ultrasonicator (Covaris, MA), end repairing, deoxyadenosine tailing, adaptor ligation and PCR amplification (10 cycles). The library with 10 pM was clustered on Illumina cBot and the flowcell was loaded on HiSeq for sequencing using Illumina 2×100 bp sequencing kit (Illumina, CA).

Bioinformatics Analysis.

RNA-Seq paired-end reads were mapped to the human hg19 genome and RefSeq transcripts and known splice junctions using an in-house, modified BWA mapping pipeline⁴³. Transcript expression levels were estimated as Fragments Per Kilobase of transcript per Million mapped reads (FPKM) and gene FPKMs were computed by summing the transcript FPKMs for each gene using the Cuffdiff program available from the Cufflinks package⁴⁴. We called a gene “expressed” in a given sample if it had a FPKM value>=0.35 based on the distribution of FPKM gene expression levels and filtered out genes that were not expressed in any sample from the final gene expression data matrix for downstream analysis. We used FPKM⁴⁴ and limma⁴⁰ to derive a gene expression profile of PAX5 p.Gly183Ser-mutated ALL compared to non-ETV6-RUNX1 B-ALL, and gene set enrichment analysis to interrogate these expression profiles, including gene sets representing previously described PAX5 activated and repressed genes¹⁶⁻¹⁹, and genes regulated during murine B lymphoid development. As one third of ETV6-RUNX1 ALL cases harbor focal PAX5 deletions (but not sequence mutations) that influence the expression of PAX5 target genes, we also incorporated gene sets of up- and down-regulated genes in PAX5 mutated ETV6-RUNX1 ALL derived from analysis of ETV6-RUNX1 mRNA-seq and WGS data. We also analyzed the overlap with the Revilla-i-Domingo et al. data and the expression differences between the familial ALL tumor samples (FAMALL) and non ETV-B-ALL wild-type for PAX5 (nonETVBALL.PAX5WT), other B-ALLs wild type for PAX5 (OtherBALL.PAX5WT), and all B-ALL cases including those with PAX5 mutations (OtherBALL)[[(Supplementary File 6).

Analysis of PAX5 in Familial ALL and Adult-Onset ALL.

PAX5 variants were absent from the sequenced remission exomes of eight other kindreds with familial childhood ALL/lymphoma and haplotype analysis in one of these families also affected with Pre-B ALL excluded the 9p13 locus. Sanger sequencing of tumor or germline in 31 additional ALL families, including four with multiple cases of non-ALL childhood malignancies revealed a single heterozygous germline variant p.Ala322Thr, which is a rare SNP rs34810717 in a child diagnosed with acute promyelocytic leukemia. In one of these families, the somatic p.Pro80Arg mutation was detected in the proband's ALL sample in association with a dic(9;20)(p11;q11.1) causing loss of the other PAX5 allele. Resequencing of PAX5 exons and untranslated regions in treatment resistant adult-onset ALLs (n=87) also showed an absence of germline PAX5 mutations.

Gene-Expression Analysis of PAX5 Transduced J558 Cells.

Gene expression profiling of J558 cells showed that one of the mutants appeared to be an outlier based on principle component analysis. Using the vectors generated with the PCA, the mean and standard deviation were calculated for the first principle component of the two-most similar wild-type and mutant samples, respectively. A sample was considered an outlier if it was greater than 10 standard deviations away from the mean (The outlier mutant sample was >44 S.D from the mean).

The genes used for analysis were constrained to those previously identified as being Pax5 targets in murine pro-B cells and mature-B cells². P-values were calculated using the binom.test function in R for a one-sided test.

The wild type PAX5 expression data was compared to PAX5 p.Gly183Ser data. Genes were denoted as up-regulated or down-regulated based on a comparison of the average of expression per condition. Genes with multiple probes on the chip were collapsed into one outcome by averaging the expression from all the probes. The activated targets of PAX5 in murine pro-B cells were significantly up-regulated when comparing the wild-type to mutant gene expression analysis. However, the repressed targets of PAX5 in pro-B cells were not significantly down regulated, when compared to the mutant (Supplemental Table 13-15).

The activated targets of PAX5 in mature-B cells were significantly up-regulated in wild-type when compared to control, in both the full set of genes as well as in the significant subset of the geneset (Supplemental Table 14). The repressed targets were significantly down-regulated over the full set of genes, but failed to achieve statistical significance when limiting to previously-identified top-targets. These data suggest that the inhibitory effect of mutant PAX5 may be comparatively diminished in mature-B cells in the gene-sets analyzed.

Activated targets of PAX5 were significantly upregulated when comparing wild type to empty vector controls for both pro-B and mature-B cell targets (Supplemental Table 15).

Additional Analysis of PAX5 Transduced J558LμM Cells and Human ALL Samples.

In addition to the GSEA analysis described in the main text, analyzed the overlap with the Revilla-i-Domingo et al. data and the expression differences between wild type PAX5, empty vector (MIR) and the PAX5 mutants, p.Gly183Ser, p.Gly183Val p.Pro80Arg, and Δ2-6 [[(Supplementary files 4 and 5)². Consistent with the G183 variants being partial hypomorphs, only subtle changes in expression of known murine PAX5 targets were detected between PAX5 wild type and mutant alleles, therefore only modest overlap was seen with the Revilla-i-Domingo et al. data (FIG. 14A). However, not all genes were regulated in the same manner as the previously defined PAX5 target gene sets. Although, J558L is a plasmacytoma cell line, SIGLEC10 is a differently regulated gene previously identified as being significantly activated in pro B cells that showed differential expression between J558LuM cells transduced with wild type PAX5 and the mutants p.Gly183Ser, p.Gly183Val p.Pro80Arg, and Δ2-6 and MIR (FIG. 14 B).

The overlap with the Revilla-i-Domingo et al. data was analyzed and the expression differences between the familial ALL tumor samples (FAMALL) and non ETV-B-ALL wild-type for PAX5 (nonETVBALL.PAX5WT), other B-ALLs wild type for PAX5 (OtherBALL.PAX5WT), and all B-ALL cases including those with PAX5 mutations (OtherBALL) (Supplementary file 6). We found 10-20 percent overlap with the Revilla-i-Domingo et al. data² (FIG. 14 C). Although, the human B-ALL samples are progenitor B cells, overall there was more overlap between genes previously identified as regulated in murine mature B cells². Two examples of differently regulated genes previously identified as being significantly regulated only in mature B cells are SCAND1 and SH3BP2 (FIG. 14 D).

In one experiment, recurrent germline susceptibility variant to pre-B ALL was not seen in germline of patients with alternate B cell malignancies. Specifically, germline DNA of six hundred and seventy patients with a history of lymphoid neoplasms (Follicular n=157, CLL/SLL n=135, DLBCL n=89, Plasma cell myeloma n=77, Hodgkin n=74, Mantle cell n=33, Marginal zone/MALT n=57, NHL NOS or T cell related n=31, Waldenström n=8, HCL n=4, Burkitt's and Burkitt's like n=5), was subjected to genotyping by a custom TaqMan assay for the PAX5 p.G183S variant. Eighty-five individuals had a family history of lymphoid malignancy in one or more first or second degree relatives. Seventeen of 275 individuals of Jewish ancestry had a family history. The PAX5 p.G183S mutation was not present in any of the germline DNA of the described cases. The absence of the mutation from the germline DNA of the entire cohort, mainly comprised of individuals with a history of mature B cell neoplasms, unselected for somatic aberrations of PAX5 or the 9p13 locus, is in keeping with the restricted pre-B ALL phenotype seen in the families reported thus far. These findings suggest PAX5 p.G183S does not represent a common founder mutation in Ashkenazim. This evidence, considered cumulatively with the incidence of the mutation in pre-B ALL described above, indicates that the PAX5 p.G183S is associated with pre-B ALL and not other B cell malignancies.

### Example 2

### Identification of Therapeutic Compounds Using Patient Materials

A method is provided for identification of therapeutic materials using patient materials. An ALL sample is obtained from a patient with the germline PAX5 variant described herein. Leukemic cells are isolated from bone marrow biopsy of the patient, the cells are expanded in vivo and in vitro, and subjected to compound screening tests. A positive control compound can be selected from a chemotherapeutic drug used for induction therapy of ALL. In some embodiments, the positive control is selected from L-asparaginase, Daunorubicin hydrochloride, or Vincristine sulfate.

Techniques for isolation and expansion of cells, and drug screening, can be performed, for example, as described by Garnett et al., 2012, Nature, 483(7391):570-575, or Naderi et al., Blood, 2009, 114(3):608-618, each of which is incorporated herein by reference.
1. Ascertainment of patient samples
2. Thaw ALL specimens, add 8 ml 10% FCS medium and spin at 2,000 rpm for 5 minutes.
3. Resuspended in 10 ml, layer over 10 ml lympho Prep and span at 2000 rpm for 45 minutes without break.
4. Harvest cells from interface, washed with 35 ml cold PBS with 1% FCS and pellet cells
5. Resuspended the cell pellet with 5 ml of IMDM medium with 10% FCS and count cells.
6. Subject 1.0×10⁷ cells to CD34 affinity column and obtain CD34+ cells.
7. Subject CD34-fraction cells to CD19 affinity column and obtain cells.
8. Set up the following in vivo and in vitro studies for each subset including.
9. Performing whole genome sequencing and genome-wide methylation analysis on DNA extracted from isolated leukemic cells.
10. Performing transcriptome sequencing made from RNA from isolated leukemic cells. Performing proteomic analysis on proteins from isolated leukemic cells.

Expansion of cells for In Vivo Study: leukemic tumor cells, each containing a PAX5 gene (coding sequence) comprising a codon 183 defined by nucleotides 547-549 of SEQ ID NO: 1 are engrafted in to a xenograft mouse model using NOD/SCID mice as in Lock et al., Blood 99, 4100-4108 (2002), which is incorporated herein by reference.

Following engraftment of the tumor, the mice would be sacrificed to retrieve human leukemia for in vitro studies and the serial transplantation into NOD/SCID or NSG mice for in vivo testing of approved therapeutics and test compounds.

Expansion of Cells for In Vitro Study:
A. Co-culture 1.0×10⁶ crude leukemic cells/MS-5 cells T-12.5 flask in IMDM with 20% FCS in duplicates with presence or absence of Flt-3/kit/IL-7 exogenous.
B. Co-culture 1.0×10⁶ CD34+ cells/MS-5 cells T-12.5 flask in IMDM with 20% FCS in duplicates with presence or absence of Flt-3/kit/IL-7 exogenous.
C. Co-culture 1.0×10⁶ CD19+/MS-5 cells T-12.5 flask in IMDM with 20% FCS in duplicates with presence or absence of Flt-3/kit/IL-7 exogenous.
D. Commercial Pre B ALL cell lines cultured in parallel.

Compound Screen.

Drugs to be screened will include a wide range of targets and processes implicated in cancer biology; targeted agents; cytotoxic chemotherapeutics, approved drugs, drugs in development and experimental tool compounds. The effect of 72 h drug treatment on; cell viability to determine drug sensitivity, including the half-maximal inhibitory concentration (IC50), and the slope of the dose-response curve as in Garnett et al., 2012., colony formation, and growth in an immunocompromised mouse model, such as the NOD/SCID or NSG mouse model.

Cell Viability Assays.

Cells will be seeded in either 96-well or 384-well microplates in medium supplemented with 5% FBS and penicillin/streptavidin. Optimal cell number will be determined to ensure each in growth phase at the end of the assay (˜70% confluency). Cells will be fixed in 4% formaldehyde for 30 min and then stained with lμM of the fluorescent nucleic acid stain Syto60 (Invitrogen) for 1 h. ALL suspension cell lines will be treated with compound immediately following plating, incubated for 72 h, and then stained with 55 μg ml⁻¹ resazurin (Sigma) prepared in glutathione-free media for 4 h. Quantification of fluorescent signal intensity will be performed using a fluorescent plate reader at excitation and emission wavelengths of 630/695 nM for Syto60, and 535/595 nM for resazurin. Measurements of cell viability during 6-day assays using threefold dilution series of olaparib will be performed in 96-well plates using Cell Titer Blue (Promega) according to the manufacturer's instructions. Measurements of cellular apoptosis will be performed using Apo-ONE caspase assay (Promega) following manufacturer's instructions as in Garnett et al., 2012.

Colony Formation Assays

Cells will be plated at low density into 35-mm cell-culture plates and the following day treated with the indicated drug concentration or vehicle control (DMSO). The medium will be changed and cells re-drugged every 3-4 days. When sufficient colonies are visible, typically after 7-21 days, cells will be washed once in PBS before fixing in ice-cold methanol for 30 min while shaking Methanol will be aspirated and Giemsa stain added at a dilution of 1:20 overnight while shaking. The following day cells will be rinsed in distilled water and air dried as in Garnett et al., 2012.

Curve Fitting of Drug Sensitivity Data

Dose-response curves will be fitted to the fluorescence signal intensities as in [ref doi:10.1038/nature11005]. Allows modeling of the heteroscedasticity in the luminescence data, and incorporation of prior knowledge of response at drug concentrations at which the data are less informative. Response curves were fitted to the fluorescence signal intensities using a Bayesian sigmoid model. Drug-response data will consist of 16 (96-well format) or 42 (384-well) drug-free positive controls, 8 (96-well) or 32 (384-well) negative (no cells) controls and drug-response points for nine half-fold concentrations. Technical replicate intensity responses will be averaged. Generalized sigmoidal response curves will be fitted as per Garnett et al., 2012.

MANOVA

As in Garnett et al., 2012, fixed effects MANOVA will be used to correlate response with genomics. An n×2 dose-response matrix consisting of IC₅₀ and slope parameter β for n cell lines will be constructed for each drug. A linear (no interaction terms) model will be used to explain observables with factors including tissue type, the mutation status of cancer genes, chromosomal re-arrangements, and MSI status. Size effects and significances will be obtained. A gene will be defined as mutated if it fulfills any of these criteria: a coding sequence variant in the cancer gene, a total copy number of 0 (homozygous deletion) or more than 7 (amplification). Genes with >1 mutated patent cell lines in the panel will be highlighted in the analysis. The effect will measure the relative difference in the mean IC50 from the wild-type to mutant group (for example, an effect of 0.1 or 10 indicates a ˜10-fold decrease or increase in drug concentration, respectively). A Benjamini-Hochberg multiple testing correction threshold with false discovery rate of 20% will be used to identify a candidate list of significant associations as in Garnett et al., 2012.

### REFERENCES CITED

1. Mullighan, C. G. et al. Genome-wide analysis of genetic alterations in acute lymphoblastic leukaemia. Nature 446, 758-64 (2007).
2. Mullighan, C. G. et al. Deletion of IKZF1 and prognosis in acute lymphoblastic leukemia. N Engl J Med 360, 470-80 (2009).
3. Kuiper, R. P. et al. High-resolution genomic profiling of childhood ALL reveals novel recurrent genetic lesions affecting pathways involved in lymphocyte differentiation and cell cycle progression. Leukemia 21, 1258-66 (2007).
4. Hemminki, K. & Jiang, Y. Risks among siblings and twins for childhood acute lymphoid leukaemia: results from the Swedish Family-Cancer Database. Leukemia 16, 297-8 (2002).
5. Pui, C. H., Robison, L. L. & Look, A. T. Acute lymphoblastic leukaemia. Lancet 371, 1030-43 (2008).
6. Mullighan, C. G. & Downing, J. R. Global genomic characterization of acute lymphoblastic leukemia. Semin Hematol 46, 3-15 (2009).
7. Mullighan, C. G. et al. BCR-ABL1 lymphoblastic leukaemia is characterized by the deletion of Ikaros. Nature 453, 110-4 (2008).
8. Nebral, K. et al. Incidence and diversity of PAX5 fusion genes in childhood acute lymphoblastic leukemia. Leukemia 23, 134-43 (2009).
9. Zhang, J. et al. Key pathways are frequently mutated in high-risk childhood acute lymphoblastic leukemia: a report from the Children's Oncology Group. Blood 118, 3080-7 (2011).
10. Nutt, S. L., Heavey, B., Rolink, A. G. & Busslinger, M. Commitment to the B-lymphoid lineage depends on the transcription factor Pax5. Nature 401, 556-62 (1999).
11. Horcher, M., Souabni, A. & Busslinger, M. Pax5/BSAP maintains the identity of B cells in late B lymphopoiesis. Immunity 14, 779-90 (2001).
12. Cobaleda, C., Jochum, W. & Busslinger, M. Conversion of mature B cells into T cells by dedifferentiation to uncommitted progenitors. Nature 449, 473-7 (2007).
13. Maier, H., Colbert, J., Fitzsimmons, D., Clark, D. R. & Hagman, J. Activation of the early B-cell-specific mb-1 (Ig-alpha) gene by Pax-5 is dependent on an unmethylated Ets binding site. Mol Cell Biol 23, 1946-60 (2003).
14. Czerny, T. & Busslinger, M. DNA-binding and transactivation properties of Pax-6: three amino acids in the paired domain are responsible for the different sequence recognition of Pax-6 and BSAP (Pax-5). Mol Cell Biol 15, 2858-71 (1995).
15. Eberhard, D., Jimenez, G., Heavey, B. & Busslinger, M. Transcriptional repression by Pax5 (BSAP) through interaction with corepressors of the Groucho family. EMBO J 19, 2292-303 (2000).
16. Pridans, C. et al. Identification of Pax5 target genes in early B cell differentiation. J Immunol 180, 1719-28 (2008).
17. Revilla, I. D. R. et al. The B-cell identity factor Pax5 regulates distinct transcriptional programmes in early and late B lymphopoiesis. EMBO J 31, 3130-46 (2012).
18. Delogu, A. et al. Gene repression by Pax5 in B cells is essential for blood cell homeostasis and is reversed in plasma cells. Immunity 24, 269-81 (2006).
19. Schebesta, A. et al. Transcription factor Pax5 activates the chromatin of key genes involved in B cell signaling, adhesion, migration, and immune function. Immunity 27, 49-63 (2007).
20. Holmfeldt, L. et al. The genomic landscape of hypodiploid acute lymphoblastic leukemia. Nat Genet 45, 242-52 (2013).
21. Dang, J., Mullighan, C. G., Phillips, L. A., Mehta, P. & Downing, J. R. Retroviral and Chemical Mutagenesis Identifies Pax5 as a Tumor Suppressor in B-Progenitor Acute Lymphoblastic Leukemia. Blood (ASH Annual Meeting Abstracts) 112, 1789-(2008).
22. Powell, B. C. et al. Identification of TP53 as an acute lymphocytic leukemia susceptibility gene through exome sequencing. Pediatr Blood Cancer (2012).
23. DePristo, M. A. et al. A framework for variation discovery and genotyping using next-generation DNA sequencing data. Nature genetics 43, 491-8 (2011).
24. Li, H. & Durbin, R. Fast and accurate short read alignment with Burrows-Wheeler transform. Bioinformatics 25, 1754-60 (2009).
25. McKenna, A. et al. The Genome Analysis Toolkit: a MapReduce framework for analyzing next-generation DNA sequencing data. Genome Res 20, 1297-303 (2010).
26. Pounds, S. et al. Reference alignment of SNP microarray signals for copy number analysis of tumors. Bioinformatics 25, 315-21 (2009).
27. Olshen, A. B., Venkatraman, E. S., Lucito, R. & Wigler, M. Circular binary segmentation for the analysis of array-based DNA copy number data. Biostatistics 5, 557-72 (2004).
28. Venkatraman, E. S. & Olshen, A. B. A faster circular binary segmentation algorithm for the analysis of array CGH data. Bioinformatics 23, 657-63 (2007).
29. Mullighan, C. G. Single nucleotide polymorphism microarray analysis of genetic alterations in cancer. Methods in molecular biology 730, 235-58 (2011).
30. Lin, M. et al. dChipSNP: significance curve and clustering of SNP-array-based loss-of-heterozygosity data. Bioinformatics 20, 1233-40 (2004).
31. Cai, Y., Brophy, P. D., Levitan, I., Stifani, S. & Dressler, G. R. Groucho suppresses Pax2 transactivation by inhibition of JNK-mediated phosphorylation. EMBO J 22, 5522-9 (2003).
32. Lundblad, A. et al. Immunochemical studies on mouse myeloma proteins with specificity for dextran or for levan. Immunochemistry 9, 535-44 (1972).
33. Sitia, R., Neuberger, M. S. & Milstein, C. Regulation of membrane IgM expression in secretory B cells: translational and post-translational events. EMBO J 6, 3969-77 (1987).
34. Maier, H. et al. Requirements for selective recruitment of Ets proteins and activation of mb-1/Ig-alpha gene transcription by Pax-5 (BSAP). Nucleic Acids Res 31, 5483-9 (2003).
35. Hombach, J., Tsubata, T., Leclercq, L., Stappert, H. & Reth, M. Molecular components of the B-cell antigen receptor complex of the IgM class. Nature 343, 760-2 (1990).
36. Team, R. D. C. R: A Language and Environment for Statistical Computing. (R Foundation for Statistical Computing, Vienna, Austria, 2009).
37. Gentleman, R. C. et al. Bioconductor: open software development for computational biology and bioinformatics. Genome Biol 5, R80 (2004).
38. Irizarry, R. A. et al. Exploration, normalization, and summaries of high density oligonucleotide array probe level data. Biostatistics 4, 249-64 (2003).
39. Johnson, W. E., Li, C. & Rabinovic, A. Adjusting batch effects in microarray expression data using empirical Bayes methods. Biostatistics 8, 118-27 (2007).
40. Smyth, G. K. Linear models and empirical bayes methods for assessing differential expression in microarray experiments. Stat Appl Genet Mol Biol 3, Article3 (2004).
41. Benjamini, Y. & Hochberg, Y. Controlling the false discovery rate: a practical and powerful approach to multiple testing. JR Stat Soc B 57, 289-300 (1995).
42. Subramanian, A. et al. Gene set enrichment analysis: a knowledge-based approach for interpreting genome-wide expression profiles. Proc Natl Acad Sci USA 102, 15545-50 (2005).
43. Zhang, J. et al. The genetic basis of early T-cell precursor acute lymphoblastic leukaemia. Nature 481, 157-63 (2012).
44. Trapnell, C. et al. Transcript assembly and quantification by RNA-Seq reveals unannotated transcripts and isoform switching during cell differentiation. Nat Biotechnol 28, 511-5 (2010).
45. Pridans, C. et al. Identification of Pax5 target genes in early B cell differentiation. J Immunol 180, 1719-28 (2008).
46. Lock, R. et al., The nonobese diabetic/severe combined immunodeficient (NOD/SCID) mouse model of childhood acute lymphoblastic leukemia reveals intrinsic differences in biologic characteristics at diagnosis and relapse. Blood 99, 4100-4108 (2002).
47. Carol, H. et al., The anti-CD19 antibody-drug conjugate SAR3419 prevents hematolymphoid relapse postinduction therapy in preclinical models of pediatric acute lymphoblastic leukemia. Clin Cancer Res 19(7), 1795-1805 (Apr. 1, 2013).

We claim:

1. A method for identifying a human subject having an elevated risk of developing pre-B cell acute lymphoblastic leukemia (ALL), said method comprising:
(a) amplifying a nucleic acid comprising a region of the PAX5 coding sequence of SEQ ID NO: 1 in a tissue sample from said human subject;
i. wherein said amplifying uses a primer set comprising a forward primer and a reverse primer, wherein said forward primer binds to a complement of said PAX 5 coding sequence at a position 5′ to codon 183 defined by nucleotides 547-549 of SEQ ID NO: 1 and wherein said reverse primer binds to said PAX 5 coding sequence 3′ to said codon 183; and
ii. wherein said amplifying generates an amplicon comprising PAX 5 codon 183; and
(b) determining in said amplicon the amino acid encoded by said codon 183;
i. wherein if said codon 183 encodes an amino acid other than glycine, said human subject has an elevated risk of developing pre-B cell ALL.

2. A method for identifying a human subject having an elevated risk of developing pre-B cell acute lymphoblastic leukemia (ALL), said method comprising:
(a) amplifying nucleic acid comprising a region of a PAX5 gene sequence of SEQ ID NO: 4 in a tissue sample from said human subject:
i. wherein said amplifying uses a primer set comprising a forward primer and a reverse primer, wherein said forward primer binds to a complement said PAX 5 coding sequence at a position 5′ to codon 183 defined by nucleotides 501-503 of SEQ ID NO: 4 and wherein said reverse primer binds to said PAX 5 coding sequence 3′ to said codon 183; and
ii. wherein said amplifying generates an amplicon comprising PAX 5 codon 183; and
(b) determining in said amplicon the amino acid encoded by said codon 183;
i. wherein if said codon 183 encodes an amino acid other than glycine, said human subject has an elevated risk of developing pre-B cell ALL.

3. The method of claim 1 wherein said determining comprises sequencing said amplicon.

4. The method of claim 1 wherein said determining comprises hybridizing a probe to said amplicon.

5. The method of claim 1 wherein said determining comprises digesting said amplicon with a restriction endonuclease that recognizes a restriction site comprising the sequence GGC (i.e. glycine) or AGC (i.e. serine) at codon 183.

6. The method of claim 5 wherein further comprising subjecting said digested amplicon to acrylamide gel or capillary electrophoresis.

7. The method of claim 1 wherein said amplifying comprises a PCR reaction.

8. The method of claim 7 wherein said PCR reaction is a digital PCR reaction.

9. The method of claim 7 wherein said PCR reaction is a digital PCR for sequence-tagged sites (STS) on chromosome 9 reaction.

10. The method of claim 8, wherein the digital PCR comprises specificity for exon 5 of PAX5.

11. The method of claim 1 wherein said primer set comprising a forward primer and a reverse primer
i. comprises a forward primer comprising a sequence selected from the group consisting of 5′-gtctgccccttcccgtag-3′ (SEQ ID NO: 5), 5′-gagcgtgattggcaggttag-3′ (SEQ ID NO: 7), 5′-gcagcgggtctcagtgtt-3′ (SEQ ID NO: 9), 5′-ctcaaagctgctccttcctg-3′ (SEQ ID NO: 11), 5′-tgcatccatgcatagtaagtagg-3′ (SEQ ID NO: 13), 5′-cagcggtgcttctcctatgt-3′ (SEQ ID NO: 15), 5′-ggccagagtagcccgttatt-3′ (SEQ ID NO: 17), 5′-ctgtgcatagctggttgagg-3′ (SEQ ID NO: 19), 5′-gggtcagtccttctcagtgc-3′ (SEQ ID NO: 21), 5′-ttggggtcaggtcctcttc-3′ (SEQ ID NO: 23), 5′-agctcagaacgtggagttgg-3′ (SEQ ID NO: 25), 5′-cgtgacaaatgtgcagaagc-3′ (SEQ ID NO: 27), 5′-acagctgcccactccataat-3′ (SEQ ID NO: 29), 5′-gactgagtgaggggaggaaa-3′ (SEQ ID NO: 31), and 5′-GCCAGAGGATAGTGGAACTTG-3′ (SEQ ID NO: 37); and
ii. comprises a reverse primer comprising a sequence selected from the group consisting of 5′-cctcctcctccagggtca-3′ (SEQ ID NO: 6), 5′-cgaagttgcaaagaacttcctc-3′ (SEQ ID NO: 8), 5′-aggcgggaaatggtgcta-3′ (SEQ ID NO: 10), 5′-tccttccggccttagtacct-3′ (SEQ ID NO: 12), 5′-gctctcaacctcttcctcca-3′ (SEQ ID NO: 14), 5′-gctctgcgtgtgaaacaaaa-3′ (SEQ ID NO: 16), 5′-cagatcttcaggaaaggcaca-3′ (SEQ ID NO: 18), 5′-cgtgtgctgaagtgttttatgc-3′ (SEQ ID NO: 20), 5′-actcgctcctctgcaggtaa-3′ (SEQ ID NO: 22), 5′-tctctgagcagaacctggtg-3′ (SEQ ID NO: 24), 5′-caccaagaagccactcttcc-3′ (SEQ ID NO: 26), 5′-ttctcagaagcgtagaggtcac-3′ (SEQ ID NO: 28), 5′-tcctaacccaccaaagcatc-3′ (SEQ ID NO: 30), 5′-agtcagacagctggaggacag-3′ (SEQ ID NO: 32); and 5′-GTGGTGAAGATGTCTGAGTAGTG-3′ (SEQ ID NO: 38); and
wherein each of said forward primer and said reverse primer is no longer than 100 nucleotides in length.

12. The method of claim 1 wherein said amplicon is from about 50 base pairs to about 1,000 base pairs in length.

13. The method of claim 1 wherein said amino acid other than glycine is serine.

14. The method of claim 1 wherein the tissue sample is a germline sample.

15. The method of claim 14 wherein the germline sample is a blood, buccal, or prenatal germline sample.

16. The method of claim 14 wherein the germline sample is obtained from a human subject exhibiting, or at risk of exhibiting, familial ALL, sporadic ALL, or a chromosome 9p cytogenetic abnormality.

17. The method of claim 1, further comprising screening a somatic sample from said subject for a chromosome 9p cytogenetic abnormality; wherein detection of the 9p cytogenetic abnormality is indicative of the need for periodic monitoring and/or treatment of ALL in the subject.

18. The method of claim 17 wherein the detecting comprises cytogenetic screening, molecular screening or hydridization of said sample.

19. The method of claim 18 wherein the hybridization is fluorescence in situ hybridization (FISH).

20. The method of claim 19 wherein the chromosome 9p cytogenetic abnormality is selected from i(9)/dic(9;v); i(9)(q10)/dic(9;v); complete or partial loss of 9p; homozygous deletion of CDKN2A/CDKN2B; copy neutral loss of heterozygosity of the germline PAX5 variant allele caused by loss of the chromosome 9p containing the wild type PAX5 allele or somatic duplication of the chromosome 9p containing the germline PAX5 variant allele.

21. The method of claim 17 wherein the somatic sample is a bone marrow sample or a tumor sample.

22. The method of claim 17 wherein the chromosome 9p cytogenetic abnormality is selected from one or more of i(9)/dic(9;v); i(9)(q10)/dic(9;v); loss of 9p; homozygous deletion of CDKN2A/CDKN2B; copy neutral loss of heterozygosity of the germline PAX5 variant allele caused by loss of the chromosome 9p containing the wild type PAX5 allele or somatic duplication of the chromosome 9p containing the germline PAX5 variant allele.

23. The method of claim 22, further comprising comparing percent of mutant allele to wild-type allele to provide a ratio.

24. The method of claim 17 wherein the cytogenetic screening is selected from digital PCR for sequence-tagged sites (STS) on chr9p, digital karyotyping, quantitative fluorescent-PCR for loss of 9p, shot-gun sequencing of free DNA for increased ratio STS on chr9p, or comparative genomic hybridization (CGH) single nucleotide polymorphism (SNP) microarray.

25. The method of claim 17 wherein the abnormality is detected in the subject, wherein the treatment of ALL in the subject comprises:
administering induction therapy to the subject.

26. A method for selecting a human embryo for implantation, the method comprising:
a. obtaining a germline sample from a human embryo; and
b. screening the germline sample by the method of claim 1;
wherein if said codon 183 encodes glycine, the embryo is selected for implantation.

27. A kit for determining the presence of a mutation in a PAX5 gene, said kit comprising:
a set of primers selected from Table 2A or Table 2B;
wherein said mutation comprises a mutation in a PAX5 gene comprising at least c.547G>A (p.Gly183Ser), in the octapeptide domain of PAX5 from SEQ ID NO:1; or a mutation in a mRNA encoding p.Gly183Ser PAX5 polypeptide of SEQ ID NO: 2.

28. The kit of claim 27 comprising a set of primers and/or probes for amplifying and/or sequencing PAX5 DNA.

29. The kit of claim 27 wherein said set of primers comprises forward primer 5′-GGGTCAGTCCTTCTCAGTGC-3′ (SEQ ID NO: 21) and reverse primer 5′-ACTCGCTCCTCTGCAGGTAA-3′ (SEQ ID NO: 22).

30. The kit of claim 27 wherein said set of primers comprises forward primer 5′-GCCAGAGGATAGTGGAACTTG-3′ (SEQ ID NO: 37) and reverse primer 5′-GTGGTGAAGATGTCTGAGTAGTG-3′ (SEQ ID NO: 38).

31. A method for identifying a candidate therapeutic compound for a human patient having (at risk of developing) pre-B cell acute lymphoblastic leukemia (ALL), said method comprising:
a. obtaining a transformed eukaryotic host cell containing a PAX 5 gene coding sequence comprising a codon 183 defined by nucleotides 547-549 (AGC) of SEQ ID NO: 1;
b. contacting said transformed eukaryotic host cell in the presence of a compound suspected of being a cancer therapeutic;
c. growing said transformed eukaryotic host cell in the absence of said compound;
d. determining the rate of growth of said host cell in the presence of said compound and the rate of growth of said host cell in the absence of said compound, and
e. comparing the growth rate of said host cells;
wherein a slower rate of growth of said host cell in the presence of said compound is indicative of a candidate therapeutic for the treatment of a human patient having (at risk of developing) pre-B cell acute lymphoblastic leukemia (ALL).

32. The method of claim 31 wherein the compound is selected from a nucleic acid, protein, antibody, or small molecule.

33. The method of claim 31 wherein the transformed eukaryotic host cell is an ALL patient derived cell.

34. The method of claim 31 wherein the rate of growth is determined by a ³H-thymidine incorporation assay, a colony forming assay, or growth in a severe combined immunodeficiency (SCID), NOD/SCID or NSG mouse model.

35. An isolated DNA comprising an altered PAX5 DNA comprising at least c.547G>A (p.Gly183Ser) in the octapeptide domain of PAX5 and at least 90% homology with the nucleotide sequence of SEQ ID NO: 1.

36. A replicative cloning vector, comprising isolated DNA of claim 30.

37. An expression system, comprising isolated DNA of claim 30 operably linked to suitable control sequences.

38. A host cell transformed with expression system of claim 32, expressing cDNA encoding PAX5 G183S.

39. A method for identifying a human subject having an elevated risk of developing pre-B cell acute lymphoblastic leukemia (ALL), wherein said human subject exhibits a chromosome 9p abnormality, said method comprising:
(a) amplifying a nucleic acid comprising a region of the PAX5 coding sequence of SEQ ID NO: 1 in a tissue sample from said human subject;
i. wherein said amplifying uses a primer set comprising a forward primer and a reverse primer, wherein said forward primer binds to a complement of said PAX 5 coding sequence at a position 5′ to codon 183 defined by nucleotides 547-549 of SEQ ID NO: 1 and wherein said reverse primer binds to said PAX 5 coding sequence 3′ to said codon 183; and
ii. wherein said amplifying generates an amplicon comprising PAX 5 codon 183; and
(b) determining in said amplicon the amino acid encoded by said codon 183;
i. wherein if said codon 183 encodes an amino other than glycine, said human subject has an elevated risk of developing pre-B cell ALL.

40. The method of claim 1 wherein the forward primer is 5′-GCCAGAGGATAGTGGAACTTG-3′ (SEQ ID NO: 37) and the reverse primer 5′-GTGGTGAAGATGTCTGAGTAGTG-3′ (SEQ ID NO: 38).

41. A primer for amplifying a nucleic acid comprising a region of PAX5, wherein the primer comprises a region of the sequence of SEQ ID NO: 37 or SEQ ID NO: 38, wherein the primer is from 8 to 200 nucleotides in length.

42. A probe for hybridizing a nucleic acid comprising a region of PAX5, wherein the probe comprises a region of the sequence of SEQ ID NO: 37 or SEQ ID NO: 38, wherein the probe is from 8 to 200 nucleotides in length, and wherein the probe is detectably labeled.

43. A kit for determining a mutation in a PAX5 protein, said kit comprising an antibody that binds to mutant p.Gly183Ser PAX5 polypeptide, having at least 75% sequence identity with SEQ ID NO: 2; wherein the antibody does not bind to p.Gly183Gly PAX5 polypeptide having at least 75% sequence identity with SEQ ID NO: 2.

44. The method of claim 11 wherein said forward primer comprises the sequence 5′-GGGTCAGTCCTTCTCAGTGC-3′ (SEQ ID NO: 21) and wherein said reverse primer comprises the sequence 5′-ACTCGCTCCTCTGCAGGTAA-3′ (SEQ ID NO: 22).


SEQUENCE LISTING (43 sequences)

SEQ ID NO: 1 - DNA, 1176 bp, Homo sapiens
  misc_feature (547)..(547): n is g or a
atggatttagagaaaaattatccgactcctcggaccagcaggacaggacatggaggagtgaatcagcttgggggggtttttgtgaatggacggccactcccggatgtagtccgccagaggatagtggaacttgctcatcaaggtgtcaggccctgcgacatctccaggcagcttcgggtcagccatggttgtgtcagcaaaattcttggcaggtattatgagacaggaagcatcaagcctggggtaattggaggatccaaaccaaaggtcgccacacccaaagtggtggaaaaaatcgctgaatataaacgccaaaatcccaccatgtttgcctgggagatcagggaccggctgctggcagagcgggtgtgtgacaatgacaccgtgcctagcgtcagttccatcaacaggatcatccggacaaaagtacagcagccacccaaccaaccagtcccagcttccagtcacagcatagtgtccactggctccgtgacgcaggtgtcctcggtgagcacggattcggccggctcgtcgtactccatcagcngcatcctgggcatcacgtcccccagcgccgacaccaacaagcgcaagagagacgaaggtattcaggagtctccggtgccgaacggccactcgcttccgggcagagacttcctccggaagcagatgcggggagacttgttcacacagcagcagctggaggtgctggaccgcgtgtttgagaggcagcactactcagacatcttcaccaccacagagcccatcaagcccgagcagaccacagagtattcagccatggcctcgctggctggtgggctggacgacatgaaggccaatctggccagccccacccctgctgacatcgggagcagtgtgccaggcccgcagtcctaccccattgtgacaggccgtgacttggcgagcacgaccctccccgggtaccctccacacgtcccccccgctggacagggcagctactcagcaccgacgctgacagggatggtgcctgggagtgagttttccgggagtccctacagccaccctcagtattcctcgtacaacgactcctggaggttccccaacccggggctgcttggctccccctactattatagcgctgccgcccgaggagccgccccacctgcagccgccactgcctatgaccgtcactga

SEQ ID NO: 2 - protein, 391 aa, Homo sapiens
  MISC_FEATURE (183)..(183): Xaa is Gly or Ser
MDLEKNYPTPRTSRTGHGGVNQLGGVFVNGRPLPDVVRQRIVELAHQGVRPCDISRQLRVSHGCVSKILGRYYETGSIKPGVIGGSKPKVATPKVVEKIAEYKRQNPTMFAWEIRDRLLAERVCDNDTVPSVSSINRIIRTKVQQPPNQPVPASSHSIVSTGSVTQVSSVSTDSAGSSYSISXILGITSPSADTNKRKRDEGIQESPVPNGHSLPGRDFLRKQMRGDLFTQQQLEVLDRVFERQHYSDIFTTTEPIKPEQTTEYSAMASLAGGLDDMKANLASPTPADIGSSVPGPQSYPIVTGRDLASTTLPGYPPHVPPAGQGSYSAPTLTGMVPGSEFSGSPYSHPQYSSYNDSWRFPNPGLLGSPYYYSAAARGAAPPAAATAYDRH

SEQ ID NO: 3 - DNA, 1001 bp, Homo sapiens
  misc_feature (501)..(501): n is c or t
ccacccctccgccctccgcacgtgtgcccctctcacggtttggccagccccctctctctggtccaaagctcctccacaaagtggacttgggtgtggagcccagcacaccctgccctcgggccgcatatctgcacgcacacagggtcttcttaaaggccaaagctcaggccacaagggctctgccaggcccgctgcttctatccaaggggtgaccatgggggaggccagcaagaatgccacagtctcaccctgctgcctgtctgtcttgcggagggctcagccctttcccgtgttcacgaaaaggacagcgtgcgggccgggggactcgctcctctgcaggtaaggggggtgttcgcgggcacctctgctgccacccgcgaccgagcgccggaaacagaccccgtggagcgcatccccgacggggctgcgcgggcctctcttaccttcgtctctcttgcgcttgttggtgtcggcgctgggggacgtgatgcccaggatgcngctgatggagtacgacgagccggccgaatccgtgctcaccgaggacacctgcgtcacggagccagtggacactgcgcggagaaagacgggcggtcagggccgcagagggctgagggcggcggaccggctgtcaccgcacgtgaggctgggggcggctgggagggagcgagcgcagggtgggcagggggcgctgaggaccctcgctctgcggaggcggccacacctgagccacgagcgcaggcctcgggcgggccgtgtgccccagttctctgcgatggggggagaaagggaagggtgggcactgagaaggactgacccctgcagaaaggaacacggggacacggggacaggaagggctttcgcttaaattccagcctatccatggatgcaggaagcaggggatggagaggagaaagaggggagccggggcccaccaacagtcagtgcttaaaaaaaaaaaaaaaaaaaaaaaaaaacctgaactactcgttaggcatttc

SEQ ID NO: 4 - DNA, 1001 bp, Homo sapiens
  misc_feature (501)..(501): n is g or a
gaaatgcctaacgagtagttcaggtttttttttttttttttttttttttttaagcactgactgttggtgggccccggctcccctctttctcctctccatcccctgcttcctgcatccatggataggctggaatttaagcgaaagcccttcctgtccccgtgtccccgtgttcctttctgcaggggtcagtccttctcagtgcccacccttccctttctccccccatcgcagagaactggggcacacggcccgcccgaggcctgcgctcgtggctcaggtgtggccgcctccgcagagcgagggtcctcagcgccccctgcccaccctgcgctcgctccctcccagccgcccccagcctcacgtgcggtgacagccggtccgccgccctcagccctctgcggccctgaccgcccgtctttctccgcgcagtgtccactggctccgtgacgcaggtgtcctcggtgagcacggattcggccggctcgtcgtactccatcagcngcatcctgggcatcacgtcccccagcgccgacaccaacaagcgcaagagagacgaaggtaagagaggcccgcgcagccccgtcggggatgcgctccacggggtctgtttccggcgctcggtcgcgggtggcagcagaggtgcccgcgaacaccccccttacctgcagaggagcgagtcccccggcccgcacgctgtccttttcgtgaacacgggaaagggctgagccctccgcaagacagacaggcagcagggtgagactgtggcattcttgctggcctcccccatggtcaccccttggatagaagcagcgggcctggcagagcccttgtggcctgagctttggcctttaagaagaccctgtgtgcgtgcagatatgcggcccgagggcagggtgtgctgggctccacacccaagtccactttgtggaggagctttggaccagagagagggggctggccaaaccgtgagaggggcacacgtgcggagggcggaggggtgg

SEQ ID NO: 5 - DNA, 18 bp, Artificial Sequence
  Primer
gtctgccccttcccgtag

SEQ ID NO: 6 - DNA, 18 bp, Artificial Sequence
  Primer
cctcctcctccagggtca

SEQ ID NO: 7 - DNA, 20 bp, Artificial Sequence
  Primer
gagcgtgattggcaggttag

SEQ ID NO: 8 - DNA, 22 bp, Artificial Sequence
  Primer
cgaagttgcaaagaacttcctc

SEQ ID NO: 9 - DNA, 18 bp, Artificial Sequence
  Primer
gcagcgggtctcagtgtt

SEQ ID NO: 10 - DNA, 18 bp, Artificial Sequence
  Primer
aggcgggaaatggtgcta

SEQ ID NO: 11 - DNA, 20 bp, Artificial Sequence
  Primer
ctcaaagctgctccttcctg

SEQ ID NO: 12 - DNA, 20 bp, Artificial Sequence
  Primer
tccttccggccttagtacct

SEQ ID NO: 13 - DNA, 23 bp, Artificial Sequence
  Primer
tgcatccatgcatagtaagtagg

SEQ ID NO: 14 - DNA, 20 bp, Artificial Sequence
  Primer
gctctcaacctcttcctcca

SEQ ID NO: 15 - DNA, 20 bp, Artificial Sequence
  Primer
cagcggtgcttctcctatgt

SEQ ID NO: 16 - DNA, 20 bp, Artificial Sequence
  Primer
gctctgcgtgtgaaacaaaa

SEQ ID NO: 17 - DNA, 20 bp, Artificial Sequence
  Primer
ggccagagtagcccgttatt

SEQ ID NO: 18 - DNA, 21 bp, Artificial Sequence
  Primer
cagatcttcaggaaaggcaca

SEQ ID NO: 19 - DNA, 20 bp, Artificial Sequence
  Primer
ctgtgcatagctggttgagg

SEQ ID NO: 20 - DNA, 22 bp, Artificial Sequence
  Primer
cgtgtgctgaagtgttttatgc

SEQ ID NO: 21 - DNA, 20 bp, Artificial Sequence
  Primer
gggtcagtccttctcagtgc

SEQ ID NO: 22 - DNA, 20 bp, Artificial Sequence
  Primer
actcgctcctctgcaggtaa

SEQ ID NO: 23 - DNA, 19 bp, Artificial Sequence
  Primer
ttggggtcaggtcctcttc

SEQ ID NO: 24 - DNA, 20 bp, Artificial Sequence
  Primer
tctctgagcagaacctggtg

SEQ ID NO: 25 - DNA, 20 bp, Artificial Sequence
  Primer
agctcagaacgtggagttgg

SEQ ID NO: 26 - DNA, 20 bp, Artificial Sequence
  Primer
caccaagaagccactcttcc

SEQ ID NO: 27 - DNA, 20 bp, Artificial Sequence
  Primer
cgtgacaaatgtgcagaagc

SEQ ID NO: 28 - DNA, 22 bp, Artificial Sequence
  Primer
ttctcagaagcgtagaggtcac

SEQ ID NO: 29 - DNA, 20 bp, Artificial Sequence
  Primer
acagctgcccactccataat

SEQ ID NO: 30 - DNA, 20 bp, Artificial Sequence
  Primer
tcctaacccaccaaagcatc

SEQ ID NO: 31 - DNA, 20 bp, Artificial Sequence
  Primer
gactgagtgaggggaggaaa

SEQ ID NO: 32 - DNA, 21 bp, Artificial Sequence
  Primer
agtcagacagctggaggacag

SEQ ID NO: 33 - DNA, 20 bp, Artificial Sequence
  Primer
cccgttattttgttgccaat

SEQ ID NO: 34 - DNA, 20 bp, Artificial Sequence
  Primer
tctgagcagaacctggtgtg

SEQ ID NO: 35 - DNA, 20 bp, Artificial Sequence
  Primer
actcacggaagaggcaaatg

SEQ ID NO: 36 - DNA, 20 bp, Artificial Sequence
  Primer
acccacctcagtgaccagac

SEQ ID NO: 37 - DNA, 21 bp, Artificial Sequence
  Primer
gccagaggatagtggaacttg

SEQ ID NO: 38 - DNA, 23 bp, Artificial Sequence
  Primer
gtggtgaagatgtctgagtagtg

SEQ ID NO: 39 - protein, 8 aa, Homo sapiens
YSINGILG

SEQ ID NO: 40 - protein, 8 aa, Homo sapiens
HSIDGILS

SEQ ID NO: 41 - protein, 8 aa, Homo sapiens
YSISGILG

SEQ ID NO: 42 - protein, 8 aa, Homo sapiens
PTQDGCQQ

SEQ ID NO: 43 - protein, 8 aa, Homo sapiens
YSINGLLG